SEQUENCE LISTING (7 sequences)

SEQ ID NO: 1 - DNA, 219 bp, Artificial Sequence
  Description of Artificial Sequence Synthetic promoter sequence
ctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgct

SEQ ID NO: 2 - DNA, 73 bp, Artificial Sequence
  Description of Artificial Sequence Synthetic promoter sequence
tcaataatggggggggggggaaagccacgttgtgtctcaaaatctctgatgttacattgcacaagataaaaat

SEQ ID NO: 3 - DNA, 69 bp, Artificial Sequence
  Description of Artificial Sequence Synthetic promoter sequence
atatcatcatgaacaataaaactgtctgcttacataaacagtaatacaaggggtgttatgagccatatt

SEQ ID NO: 4 - DNA, 314 bp, Escherichia coli
gaattcccgcagattaacgcgcataacaagcggtttactcgttttggcctgcaatgtaacataatatacattatgcgcactaaggtagaggcagcaagattatgcggttttgatcagaactcagttaaccatcccgggattctgctctggcccattcagcgcagttttttactttggatgaagttaacccatgttatattgcacaagataaaaatatatcatcatgaacaataaaactgtctgcttacataaacagtaatacaaggggtgttatgagccatattcaacgggaaacgtcttgctcgagggaattc

SEQ ID NO: 5 - DNA, 3094 bp, Escherichia coli
ggctttgttgaataaatcagatttcgggtaagtctcccccgtagcgggttgtgttttcaggcaatacgcacgctttcaggcatacctgctttcgtcattttgttcagcgctcgtaccagggccatagcctccgcaacctgaccatcgtagtcacgcagcgtcagtgaacccccgaacagctgttttacccggtacatcgccgtttccgctatcgagcgacggttgtaatctgttgtccatttccaccgcgcattactcccggtcattcgctgattagccactgcacggttacggtctgcatattcaccgggccagtaacccgcaccttttcggggagggataagcgcgctgattttcttacgccgcagttcatcgtgacagagccgggtgtcgtaagcgccgtctgccgatgctgccctgatttttctgtgagtctgccggataagacccgggaaggcttctgagtcggtcacattgttcagcgacaggtcagcgcagatgatttcatgtgttttactgtcaacggcgagatgcagcttacgccagatacggcggcgttcctggccatgctttttgactttccattcgccttcaccaaagaccttcagcccggtggaatcaatcaccagatgcgcgatttcaccccgggtgaacgttttgaaactgatattaaccgactttgcccgcctgctgacacagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagctgtgtcagcaggcgggcaaagtcggttaatatcagtttcaaaacgttcacccggggtgaaatcgcgcatctggtgattgattccaccgggctgaaggtctttggtgaaggcgaatggaaagtcaaaaagcatggccaggaacgccgccgtatctggcgtaagctgcatctcgccgttgacagtaaaacacatgaaatcatctgcgctgacctgtcgctgaacaatgtgaccgactcagaagccttcccgggtcttatccggcagactcacagaaaaatcagggcagcatcggcagacggcgcttacgacacccggctctgtcacgatgaactgcggcgtaagaaaatcagcgcgcttatccctccccgaaaaggtgcgggttactggcccggtgaatatgcagaccgtaaccgtgcagtggctaatcagcgaatgaccgggagtaatgcgcggtggaaatggacaacagattacaaccgtcgctcgatagcggaaacggcgatgtaccgggtaaaacagctgttcgggggttcactgacgctgcgtgactacgatggtcaggttgcggaggctatggccctggtacgagcgctgaacaaaatgacgaaagcaggtatgcctgaaagcgtgcgtattgcctgaaaacacaacccgctacgggggagacttacccgaaatctgatttattcaacaaagcc

SEQ ID NO: 6 - DNA, 4176 bp, Artificial Sequence
  Description of Artificial Sequence Synthetic vector
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 7 - DNA, 3236 bp, Escherichia coli
tacccaaggtcgctggtggtgatttcgccgccaactaaaaccaatgccggtttttacgtaggtttcgcaagcaacgcgtgctttcggatcctgttcgaggatcgcgtctaaaacggcatcagaaatttggtcagcaattttgtcaggatgcccttcagagacggactcggacgtaaaaaggtgttttgccatatttaatatcacctaaagagaatttggttagctcaaactgttgtgtggattttctgtggtagcggatcctaccacgactctgcaggttaaaaacactggcagtctgagtgttaatcggtatggatggattaacatctggatggctattttaggtcaattcttcaccctatttccacttttttttgaatcgtgtctcattctgttaaaaacgtggctggaaatttttcctgacaatgccggcattctgcgtatttatcttttgcaattttctgccattgtggggtataaaacgcggcgcgcggcttaaataaaaagcacacgacgtttctttcgtgttgccacttccagccgggttcaaatcagagttttggcttgtgggttcgtcttaacaggcggccgtggaggtgatacgaaataatgaaccgttgtctgctgcttaacctgtctcaccgttctggtgaagattcgttccccgcactctgcatctctgctttgcatacctgccgatgttatacccatctcggcgcttctcaggattcaagagctggttacagttactgaggactgaacaagggcgctcttgtaaaaacaagagttttctcgtggtttcgccgaactttcacacttacgttcggttatgtgcttaataatgttatgaaaaagaaaccggttgcgcagttggagcgtcagcattcactgctggaaaatccatgtgcttatgggttgttatcgcagttccaggctgcgatagtcgttaactgttttacacttaataaaataatttgaggttcgctatgtctgacgacatgtctatgggtttgccttcgtcagcgggcgaacacggtgtactacgctccatgcaggaggttgcaatgagctcccaggaagccagcaagatgctgcgtacttacaatattgcctggtggggcaataactactatgacgttaacgagctgggccacattagcgtgtgcccggacccggacgtcccggaagctcgcgtcgatctcgcgcagttagtgaaaactcgtgaagcacagggccagcgtctgcctgcactgttctgtttcccacagatcctgcagcaccgtttgcgttccattaacgccgcgttcaaacgtgcgagggaatcctacggctataacggcgattacttccttgtttatccgatcaaagttaaccagcaccgccgcgtgattgagtccctgattcattcgggcgaaccgctgggtctggaagccggttccaaagccgagttgatggcagtactggcacatgctggcatgacccgtagcgtcatcgtctgcaacggttataaagaccgcgaatatatccgcctggcattaattggcgagaagatggggcacaaggtctatctggtcattgagaagatgtcagaaatcgccattgtgctggatgaagcagaacgtctgaatgtcgttcctcgtctgggcgtgcgtgcacgtctgcgttcgcagggttcgggtaaatggcagtcctccggcggggaaaaatcgaagttcggcctggctgcgactcaggtactgcaactggttgaaaccctgcgtgaagccgggcgtctcgacagcctgcaactactgcacttccacctcggttcgcagatggcgaatattcgcgatatcgcgacaggcgttcgtgaatccgcgcgtttctatgtggaactgcacaagctgggcgtcaatattcagtgcttcgacgtcggcggcggtctgggcgtggattatgaaggtactcgttcgcagtccgactgttcggtgaactacggcctcaatgaatacgccaacaacattatctgggcgattggcgatgcgtgtgaagaaaacggtctgccgcatccgacggtaatcaccgaatcgggtcgtgcggtgactgcgcatcacaccgtgctggtgtctaatatcatcggcgtggaacgtaacgaatacacggtgccgaccgcgcctgcagaagatgcgccgcgcgcgctgcaaagcatgtgggaaacctggcaggagatgcacgaaccgggaactcgccgttctctgcgtgaatggttacacgacagtcagatggatctgcacgacattcatatcggctactcttccggcatctttagcctgcaagaacgtgcatgggctgagcagctttatttgagcatgtgccatgaagtgcaaaagcagctggatccgcaaaaccgtgctcatcgtccgattatcgacgagctgcaggaacgtatggcggacaaaatgtacgtcaacttctcgctgttccagtcgatgccggacgcatgggggatcgaccagttgttcccggttctgccgctggaagggctggatcaagtgccggaacgtcgcgctgtgctgctggatattacctgtgactctgacggtgctatcgaccactatattgatggtgacggtattgccacgacaatgccaatgccggagtacgatccagagaatccgccgatgctcggtttctttatggtcggcgcatatcaggagatcctcggcaacatgcacaacctgttcggtgataccgaagcggttgacgtgttcgtcttccctgacggtagcgtagaagtagaactgtctgacgaaggcgataccgtggcggacatgctgcaatatgtacagctcgatccgaaaacgctgttaacccagttccgcgatcaagtgaagaaaaccgatcttgatgctgaactgcaacaacagttccttgaagagttcgaggcaggtttgtacggttatacttatcttgaagatgagtaagtcctgtgttacttgaatccgcttaatttagcggtgataatccgccacaatttattgtgacaaatccaacccttcctcgtcgggcctaacgacgcggaagggtttttttatatcgactttgtaataggagtccatccatgagcaccttaggtcatcaatacgataactcactggtttccaatgcctttggttttttacgcctgccgatgaacttccagccgtatgacagcgatgcagactgggtgattactggcgtgccgttcgatatggcca

(19) United States
(12) Patent Application Publication
(10) Pub. No.: US 2008/0274137 A1
(43) Pub. Date: Nov. 6, 2008

(54) DNA plasmids having improved expression and stability

(76) Inventor: Jean Christophe Francis Audonnet, Lyon (FR)

Correspondence Address: Judy Jarecki-Black, Ph.D.,J.D, 3239 Satellite Blvd., Duluth, GA, 30096, US

(21) Appl. No.: 11/799,520

(22) Filed: May 2, 2007

(51) Int. Cl. A61K 39/12; C07H 21/02; C12N 15/00

(52) U.S. Cl. 424/204.1; 435/320.1; 536/23.1

(57) ABSTRACT

The present invention relates to compositions and methods to improve expression of exogenous polypeptides, such as an antigen, epitope, immunogen, peptide or polypeptide of interest. More particularly, the present invention provides for DNA plasmids with increased expression and stability in compositions and methods useful for DNA vaccines.

### INCORPORATION BY REFERENCE

This application makes reference to U.S. Provisional Application 60/795,324 filed Apr. 27, 2006. All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates generally to DNA vaccines and methods of using the same. More particularly, the present invention relates to DNA plasmids with improved expression and stability useful for DNA vaccines.

### BACKGROUND OF THE INVENTION

DNA vaccines, also referred to as genetic, plasmid or polynucleotide vaccines, represent a relatively simple and economical method to exploit gene transfer for immunization against antigens. The low toxicity associated with DNA vaccines favors its further development, but additional strategies to improve the potency of this approach are needed if it is to be successfully integrated into the clinical setting (reviewed by Shaw & Strong, Front Biosci. 2006 Jan. 1; 11:1189-98). DNA vaccination can overcome most disadvantages of conventional vaccine strategies and has potential for vaccines of the future. However, a commercial product still has not reached the market. One possible explanation could be the technique's failure to induce an efficient immune response in humans, but safety may also be a fundamental issue (reviewed by Glenting & Wessels, Microb Cell Fact. 2005 Sep. 6; 4:26).

The pVR1020 or 1012 plasmid (VICAL Inc.; Luke C. et al., Journal of Infectious Diseases, 1997, 175, 91-97; Hartikka J. et al., Human Gene Therapy, 1996, 7, 1205-1217, see, e.g., U.S. Pat. Nos. 5,846,946; 6,451,769; 6,586,409 and 6,875,748) is a DNA plasmid vector utilized for the insertion of a polynucleotide sequence. The pVR1020 plasmid is derived from pVR1012 and contains the human tPA signal sequence. Additional DNA plasmids are found, for example, in U.S. Pat. Nos. 6,852,705; 6,818,628; 6,586,412; 6,576,243; 6,558,674; 6,464,984; 6,451,770; 6,376,473 and 6,221,362. However, there are disadvantages with the pVR1012-based plasmids for DNA vaccination, such as plasmid instability and copy-number heterogeneity.

Accordingly, there is a need for an effective DNA vaccine, especially with respect to expression of a target antigen, epitope, immunogen, peptide or polypeptide of interest in an amount sufficient to elicit a protective response.

Citation or identification of any document in this application is not an admission that such document is available as prior art to the present invention.

### SUMMARY OF THE INVENTION

The invention is based, in part, on an experimental observation that insertion of a transposon between a kanamycin resistance gene promoter and translation initiation abolished kanamycin resistance, as expected, and unexpectedly, enabled high rates of plasmid replication. In other words, plasmid yields of the mutated form (containing the transposon) were three times higher than the parental plasmid yields. It was therefore hypothesized that expression efficiency of the kanamycin resistance (“KanaR”) gene has an impact on plasmid replication in bacteria, i.e., decreased kanamycin resistance expression leads to increased plasmid replication rates. Specifically, the high KanaR expression rate may represent a strong metabolic burden, which interferes with optimal plasmid replication rate and/or KanaR gene transcription interferes with the replication origin (ORI).

The present invention relates to a DNA plasmid which may comprise a kanamycin resistance (“KanaR”) gene wherein the kanamycin resistance gene comprises a less effective promoter for KanaR expression and/or a less efficient initiation codon. In an advantageous embodiment, the KanaR promoter is the P1 promoter. The resultant DNA plasmid results in higher plasmid yields and higher plasmid stability presumably due to decreased KanaR expression. Advantageously, the DNA plasmid may be pLL10 or pLL14.

The present invention also relates to a DNA plasmid which may comprise a kanamycin resistance gene wherein a transposon is inserted between the KanaR promoter and translation initiation, thereby resulting in decreased KanaR expression. The resultant DNA plasmid results in higher plasmid yields and higher plasmid stability presumably due to decreased KanaR expression.

The present invention encompasses formulations for delivery and expression of an antigen, epitope, immunogen, peptide or polypeptide of interest, wherein the formulation may comprises any of the DNA plasmids disclosed herein and a pharmaceutically or veterinarily acceptable carrier, vehicle or excipient. In an advantageous embodiment, the carrier, vehicle or excipient may facilitate transfection and/or improves preservation of the vector or protein. Advantageously, the antigen, epitope, immunogen, peptide or polypeptide of interest may be derived from an avian, bovine, canine, equine, feline or porcine virus or pathogen.

The invention further provides for methods of stimulating an immune response in an animal which may comprise administering an effective amount of the formulations disclosed herein to cells of the animal and expressing the antigen, epitope, immunogen, peptide or polypeptide of interest in the cells. The invention also provides for methods of eliciting an immune response in an animal which may comprise administering an effective amount of the formulations disclosed herein to cells of the animal and expressing the antigen, epitope, immunogen, peptide or polypeptide of interest in the cells. Advantageously, the animal may be an avian, a bovine, a canine, an equine, a feline or a porcine.

The invention also encompasses kits for performing any one of the methods of described above which may comprise the DNA plasmid or formulations of disclosed herein plus instructions for performing the methods of stimulating or eliciting an immune response in an animal.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 depicts the three potential promoters of the KanaR expression cassette as identified in Example 1: P1 (SEQ ID NO: 1), P2 (SEQ ID NO: 2) and P3 (SEQ ID NO: 3). P2 and P3 partially overlap;

FIG. 2 depicts a schematic of pVR1012, illustrating the direction of KanaR transcription and the position of the replication origin, ORI;

FIG. 3 illustrates how the KanaR cassette of plasmid pVR1012 in Example 1 was cloned into the mutagenesis vector pALTER-1;

FIG. 4 depicts how a PacI restriction site in Example 1 was introduced downstream of the KanaR ORF, producing plasmid pLL2, a SwaI restriction site was introduced upstream, a RsrII restriction site was introduced downstream, producing pLL4, and finally the translation initiator codon ATG was mutated to TTG, producing pLL6;

FIG. 5 depicts how the mutated KanaR cassette of pLL4 in Example 1 was cloned into plasmid pVR1012, producing pLL7;

FIG. 6 depicts how the rrnB terminator sequence identified in Example 1 was cloned into plasmid pLL7 by PCR from pSB062 and subsequent PacI RsrII digestion to form pLL9;

FIG. 7 depicts the cloning of the speA terminator sequence, identified in Example 1, into plasmid pLL7 by annealing of synthetic oligonucleotides and PacI RsrII digestion to form pLL11;

FIG. 8 depicts how the mutated translation initiation sequence of plasmid pLL6 was cloned into pLL9 by MscI PacI digestion to form pLL10;

FIG. 9 illustrates how the promoter sequences P2/P3 (SEQ ID NOS: 2-3) with P1 (SEQ ID NO: 1), P1 (SEQ ID NO: 1), and P2/P3 (SEQ ID NOS: 2-3), which were identified in Example 1, were cloned by URS11-URS12 PCR, URS13-URS12 PCR and URS11-URS14 PCR, respectively;

FIG. 10 depicts how promoter sequence P2/P3 (SEQ ID NOS: 2-3) with P1 (SEQ ID NO: 1) was cloned into plasmid pLL9 to form pLL13;

FIG. 11 depicts how promoter sequence P1 (SEQ ID NO: 1) was cloned into plasmid pLL9 to form pLL14;

FIG. 12 depicts how promoter sequence P2/P3 (SEQ ID NOS: 2-3) was cloned into plasmid pLL9 to form pLL15;

FIG. 13 depicts how promoter sequence P1 (SEQ ID NO: 1) was cloned into plasmid pLL7 to form pLL16;

FIG. 14 depicts several pVR1012 derivatives generated with the various suitable promoters and terminators identified in Example 1. The pLL13, pLL14, pLL15 and pLL16 constructs have a 192 base pair deletion between the CMV promoter and the Kana P1 (SEQ ID NO: 1) promoter;

FIG. 15 depicts a schematic of pVR1012 derivatives, illustrating the 192 base pair region deleted in the pLL13 to pLL16 constructs of Example 1;

FIG. 16 depicts two graphs of the ability of bacterial colonies to resist increasing Kanamycin concentrations, according to Example 1, at 50 μl and 100 μl bacterial suspension dilutions, respectively;

FIG. 17 depicts graphs of the plasmid yields obtained under laboratory growth conditions. The bacterial colonies of Example 1 were grown in liquid LB medium with Kana 100 μg/ml;

FIG. 18 depicts plasmid yields expressed as a ratio to the pLL14 plasmid yield (baseline=100). Experiment 1 depicts 8 to 15 clones per construct, Experiment 2 depicts 6 clones for pVR1012, 2 for pLL14 and 25 for pLL16 and Experiment 3 (25 clones for pVR1012, pPB662 and pLL14, and 20 for pLL19);

FIG. 19 depicts graphs of the plasmid yields obtained under laboratory growth conditions. The bacterial colonies were grown in EGLI medium. Plasmid yields are expressed as a ratio to the pVR1012 parental plasmid yield (baseline=100) and

FIG. 20 depicts individual plasmid yield (indexed on pVR1012 construct average) showing clone heterogeneity. (*: mutated plasmid) and depicts graphs of the plasmid yields in three different construct EGLI-adapted clones according to Example 1. Variability is expressed as an index, where the first clone is considered the 100% reference.

### DETAILED DESCRIPTION

The invention is based, in part, on an experimental observation that insertion of a transposon between a kanamycin resistance gene promoter and translation initiation abolished kanamycin resistance, as expected, and unexpectedly, enabled high rates of plasmid replication. In other words, plasmid yields of the mutated form (containing the transposon) were three times higher than the parental plasmid yields. It was therefore hypothesized that expression efficiency of the kanamycin resistance (“KanaR”) gene has an impact on plasmid replication in bacteria, i.e., decreased kanamycin resistance expression leads to increased plasmid replication rates. Specifically, in the context of nutrient limitation, the high KanaR expression rate may represent a strong metabolic burden, which interferes with optimal plasmid replication rate and/or KanaR gene transcription interferes with the replication origin (ORI).

The invention encompasses any contemplated DNA plasmid wherein KanaR expression is decreased, thereby resulting in increased plasmid yields and higher plasmid stability as compared to, for example, the pVR1012 plasmid or a derivative thereof, without reduced KanaR expression.

The present invention relates to a DNA plasmid which may comprise KanaR wherein the kanamycin resistance gene comprises a less effective promoter for KanaR expression and/or a less efficient initiation codon. In an advantageous embodiment, the KanaR promoter is the P1 promoter and the initiation codon is ATG. The resultant DNA plasmid results in higher plasmid yields and higher plasmid stability presumably due to decreased KanaR expression. Advantageously, the DNA plasmid may be pLL10 or pLL14.

The present invention also relates to a DNA plasmid which may comprise a kanamycin resistance gene wherein a transposon is inserted between the KanaR promoter and translation initiation, thereby resulting in decreased KanaR expression. The resultant DNA plasmid results in higher plasmid yields and higher plasmid stability presumably due to decreased KanaR expression.

The expression vector is a plasmid vector or a DNA plasmid vector, in particular an in vivo expression vector. In a specific, non-limiting example, the DNA plasmid vector contains elements of the pVR1020 or 1012 plasmid (VICAL Inc.; Luke C. et al., Journal of Infectious Diseases, 1997, 175, 91-97; Hartikka J. et al., Human Gene Therapy, 1996, 7, 1205-1217, see, e.g., U.S. Pat. Nos. 5,846,946; 6,451,769; 6,586,409 and 6,875,748). The pVR1020 plasmid is derived from pVR1012 and contains the human tPA signal sequence. In one embodiment the human tPA signal comprises from amino acid M(1) to amino acid S(23) in Genbank under the accession number HUMTPA14. In another specific, non-limiting example, the plasmid utilized as a vector for the insertion of a polynucleotide sequence can contain the signal peptide sequence of equine IGF1 from amino acid M(24) to amino acid A(48) in Genbank under the accession number U28070. Additional information on DNA plasmids which may be consulted or employed in the practice are found, for example, in U.S. Pat. Nos. 6,852,705; 6,818,628; 6,586,412; 6,576,243; 6,558,674; 6,464,984; 6,451,770; 6,376,473 and 6,221,362.

In another embodiment, the plasmid may be any plasmid with a kanamycin resistance gene including, but not limited to, Clontech vectors Living Colors pAcGFP1, Living Colors pAcGFP1-C1, Living Colors pAcGFP1-N1, pAmCyan1-C1, pAmCyan1-N1, pAsRed2-C1, pAsRed2-N1, pCMV-DsRed-Express, pDsRed2-1, pDsRed2-C1, pDsRed2-N1, pDsRed-Express-1, pDsRed-Express-C1, pDsRed-Express-N1, pDsRed-Monomer-C1, pDsRed-Monomer-N1, pHcRed1-1, pHcRed1-C1, pHcRed1-N1/1, pIRES2-DsRed2, pIRES2-DsRed-Express, pLPS-AcGFP1-N Acceptor, Proteasome Sensor, pTimer, pZsGreen1-1, pZsGreen1-C1, pZsGreen1-N1, pZsYellow1-C1 and pZsYellow1-N1 Vector; Invitrogen vectors pCR3, pCR3.1-Uni, pCR1000 and pZErO-2.1 and Stratagene pCMV-3 Tag, pBK-CMV Phagemid, ZAP Express, pCMV-Script, pCMV-Tag, PCR-Script pMC1neo and pMC1neo Poly A vectors. Reference is also made to U.S. Pat. Nos. 6,942,975, 6,887,702, 6,849,442, 6,846,970, 6,825,012, 6,806,399, 6,803,230, 6,790,607, 6,696,278, 6,667,150, 6,624,344, 6,620,990, 6,610,909, 6,585,976, 6,573,437, 6,570,067, 6,562,584, 6,528,703, 6,503,748, 6,475,731, 6,410,317, 6,410,314, 6,387,654, 6,376,744, 6,350,934, 6,303,383, 6,297,054, 6,284,541, 6,258,999, 6,255,560, 6,248,937, 6,235,518, 6,174,708, 6,127,171, 6,121,511, 6,117,651, 6,054,635, 6,037,524, 6,018,103, 5,994,625, 5,981,191, 5,981,182, 5,977,439, 5,925,544, 5,910,488, 5,866,404, 5,851,808, 5,851,804, 5,830,727, 5,824,877, 5,792,935, 5,783,394, 5,750,871, 5,733,753, 5,733,744, 5,731,179, 5,723,746, 5,716,803, 5,712,112, 5,705,361, 5,654,180, 5,599,670, 5,591,577, 5,589,623, 5,569,834, 5,567,599, 5,565,347, 5,504,005, 5,463,174, 5,460,952, 5,436,138, 5,416,250, 5,416,011, 5,378,618, 5,256,568, 5,169,755, 5,137,829, 5,053,335, 5,004,863, 4,935,340, 4,920,054, 4,795,855, 4,782,022, 4,771,002, 4,626,510, and 4,567,146.

The present invention also encompasses plasmids with other antibiotic resistance genes in addition to kanamycin resistance, such as but not limited to ampicillin, chloramphenicol, neomycin and tetracycline resistance.

In one embodiment, the plasmid may be any plasmid with an ampicillin resistance gene including, but not limited to, Clontech vectors Living Colors pAcGFP1, pAmCyan, pAsRed2, pbetaga1-Basic, pbetaga1-Control, pBI Tet, pBI-G Tet, pBI-L Tet, pCMVbeta, pCMV-Myc & pCMV-HA, pDsRed2, pDsRed-Express, pDsRed-Monomer Vector, pGFP, pGFPuv, pHcRed1, pIRES, pIRESbleo3, pIRESneo3, pIRESpuro3, pLP-IRESneo Acceptor, pLP-LNCX Acceptor, pLP-TRE2 Acceptor, pPUR, pRevTet-Off, pRevTet-Off-IN, pRevTet-On, pSEAP2-Basic, pSEAP2-Control, pTet-Off, pTet-On, pTet-tTS, pTimer, pTimer-1, pTK-Hyg, pTRE2, pTRE2hyg, pTRE2hyg2-6xHN, pTRE2hyg2-HA, pTRE2hyg2-Myc, pTRE2pur, pTRE2pur-6xHN, pTRE2pur-HA, pTRE2pur-Myc, pTRE-6xHN, pTRE-HA, pTRE-Myc, pTRE-Tight, pTRE-Tight-DsRed2, pZsGreen and pZsYellow; Invitrogen vectors p2Bac, p2Bac/CAT/CAT, pAc5.1/V5-His, pAc5.1/V5-His/LacZ, pBAD-TOPO, pBlueBac, pBlueBac/His, pBlueBac/His/CAT, pBlueBac2, pBlueBac3, pBlueBac4, pBlueBac4/CAT, pcDNA1/Amp, pcDNA3/CAT, pcDNAII, pCMVSport1, pCMVSport2, pCMVSport2.2, pCMVSport4, pCR3, pCR3.1-Uni, pCRBac, pCRT7/CT-LacZ, pCRT7/CT-TOPO, pCRT7/NT-E3, pCRT7/NT-TOPO, pDW232, pFastBac1, pMEP4, pMT/LacZ, pMT/V5-His-TOPO, pPIC3, pPIC3K, pRBK, pSL301, pSPORT1-CAT, pYesTrp2-RalGDS and pYesTrp2-RalGDS and Stratagene PCR-Script, pBC Phagemid, pMC1neo and pMC1neo Poly A, pBlueScript II Phagemid and SuperCos I vectors. Reference is also made to U.S. Pat. Nos. 6,987,006, 6,972,322, 6,916,611, 6,887,702, 6,803,230, 6,790,607, 6,706,524, 6,696,278, 6,667,150, 6,569,678, 6,562,584, 6,544,782, 6,521,449, 6,503,748, 6,486,134, 6,410,317, 6,410,314, 6,387,654, 6,376,192, 6,291,238, 6,255,071, 6,221,630, 6,140,087, 6,127,171, 6,025,193, 6,025,192, 6,010,875, 5,981,279, 5,981,182, 5,955,363, 5,925,544, 5,919,676, 5,912,153, 5,894,060, 5,877,400, 5,866,404, 5,851,808, 5,834,191, 5,830,727, 5,830,690, 5,786,162, 5,776,773, 5,773,697, 5,766,940, 5,733,753, 5,731,193, 5,716,803, 5,705,361, 5,691,155, 5,602,300, 5,527,691, 5,504,005, 5,470,729, 5,436,138, 5,434,065, 5,432,082, 5,378,618, 5,290,691, 5,264,354, 5,256,568, 5,256,546, 5,160,489, 5,151,364, 5,147,789, 5,143,836, 5,126,252, 5,093,251, 5,081,022, 4,997,767, 4,988,622, 4,935,364, 4,920,054, 4,889,806, 4,879,230, 4,876,202, 4,845,031, 4,808,519, 4,766,072, 4,752,574, 4,703,012, 4,634,678, 487,835, 4,349,629 and 4,340,674.

In another embodiment, the plasmid may be any plasmid with a chloramphenicol resistance gene including, but not limited to, Clontech vectors pDNR-LacZ Donor Reporter, pDNR-SEAP Donor Reporter, pLP-AcGFP1-C Acceptor, pLP-BacPAK9 Acceptor, pLP-BacPAK9-6xHN Acceptor, pLP-CMV-HA Acceptor, pLP-CMV-Myc Acceptor, pLP-CMVneo Acceptor, pLP-GADT7 AD Acceptor, pLP-GBKT7 DNA-Acceptor, pLP-IRESneo Acceptor, pLP-LNCX Acceptor, pLP-PROTet-6xHN Acceptor, pLP-RevTRE Acceptor, pLPS-AcGFP1-N Acceptor and pLP-TRE2 Acceptor; Invitrogen vectors pLysS and pSPORT1-CAT and Stratagene pBC Phagemid and PCR-Script vectors. Reference is also made to U.S. Pat. Nos. 6,916,611, 6,900,010, 6,887,702, 6,884,576, 6,846,671, 6,803,230, 6,696,278, 6,673,537, 6,667,150, 6,562,584, 6,548,246, 6,503,748, 6,436,694, 6,420,110, 6,410,317, 6,410,314, 6,391,640, 6,387,654, 6,376,192, 6,331,527, 6,309,883, 6,309,830, 6,291,211, 6,255,071, 6,252,140, 6,221,630, 6,146,871, 6,127,171, 6,107,093, 6,083,750, 6,031,151, 6,025,192, 6,001,564, 5,994,132, 5,994,066, 5,981,182, 5,955,363, 5,932,479, 5,928,891, 5,925,544, 5,925,523, 5,908,747, 5,866,404, 5,851,808, 5,821,093, 5,766,940, 5,733,753, 5,728,571, 5,716,803, 5,707,830, 5,639,644, 5,591,577, 5,470,729, 5,437,988, 5,434,065, 5,256,568, 5,256,546, 5,053,335, 5,004,863, 4,868,111, 4,839,284, 4,752,574 and 4,711,849.

In another embodiment, the plasmid may be any plasmid with a neomycin resistance gene including, but not limited to, Clontech vectors pRevTet-Off Vector, pRevTet-Off-IN Vector, pIRES Vector, pIRESbleo3 Vector, pIREShyg3 Vector, pIRESneo3 Vector, pIRESpuro3 Vector, pLP-IRESneo Acceptor Vector, pTet-Off Vector, pTet-On Vector, pIRES2-DsRed-Express Vector, pLXIN Retroviral Vector, pIRES2-DsRed2 Vector, Proteasome Sensor Vector, pLXSN Retroviral Vector, pCMV-DsRed-Express Vector, Living Colors pAcGFP1-C1 Vector, pAmCyan1-C1 Vector, pAsRed2-C1 Vector, pDsRed2-C1 Vector, pDsRed-Express-C1 Vector, pDsRed-Monomer-C1 Vector, pHcRed1-C1 Vector, pZsGreen1-C1 Vector, pZsYellow1-C1 Vector, Living Colors pAcGFP1 Vector, pDsRed2-1 Vector, pDsRed-Express-1 Vector, pHcRed1-1 Vector, pTimer Vector, pZsGreen1-1 Vector, pQCXIX Retroviral Vector, LRCX Retroviral Vector Set, pLNCX2 Retroviral Vector, pLP-LNCX Acceptor Vector, pQCXIN Retroviral Vector, Living Colors pAcGFP1-N1 Vector, pAmCyan1-N1 Vector, pAsRed2-N1 Vector, pDsRed2-N1 Vector, pDsRed-Express-N1 Vector, pDsRed-Monomer-N1 Vector, pHcRed1-N1/1 Vector, pLPS-AcGFP1-N Acceptor Vector, pZsGreen1-N1 Vector, pZsYellow1-N1 Vector and VP16 Minimal Domain Vector Set; Invitrogen vectors pcDNA1/Neo, pcDNA3/CAT (replaced with pcDNA3.1/CAT), pCR3 (replaced with pCR3.1), pCR3.1-Uni and pRc/CMV and Stratagene pCMV-3Tag Vectors, pCMV-Script Vector, SuperCos I Vector, ZAP Express Vector, pCMV-Tag Vectors, pMC1neo and pMC1neo Poly A Vectors, PCR-Script Cloning Kits, Vitality hrGFP Mammalian Expression Vectors, pBK-CMV Phagemid Vector and Lambda ZAP-CMV Vector. Reference is also made to U.S. Pat. Nos. 7,026,525, 6,942,995, 6,905,818, 6,815,185, 6,806,399, 6,787,687, 6,747,189, 6,673,602, 6,673,537, 6,667,150, 6,624,344, 6,620,990, 6,440,444, 6,429,357, 6,391,633, 6,376,192, 6,316,253, 6,294,187, 6,291,211, 6,255,560, 6,252,140, 6,248,937, 6,232,526, 6,221,630, 6,210,930, 6,207,879, 6,194,636, 6,114,146, 6,096,717, 6,071,512, 5,998,144, 5,985,560, 5,969,211, 5,955,363, 5,955,319, 5,939,288, 5,902,577, 5,894,060, 5,891,634, 5,830,698, 5,807,742, 5,750,871, 5,733,779, 5,665,565, 5,639,663, 5,614,381, 5,583,278, 5,470,726, 5,463,174, 5,416,011, 5,352,605, 5,278,056, 5,256,568, 5,149,636, 5,017,478, 4,957,865, 4,935,340, 4,839,284, 4,792,520, 4,766,066, 4,752,574, 4,740,463, 4,663,285, 4,536,475, 4,513,086, 4,513,085, 4,503,155, 4,468,462, 4,460,688, 4,430,434 and 4,416,994.

In another embodiment, the plasmid may be any plasmid with a tetracycline resistance gene including, but not limited to, Clontech vectors pRevTet-Off Vector, pRevTet-On Vector, pRevTet-Off-IN Vector, Creator-Compatible PROTet 6xHN Bacterial Expression System, PROTet 6xHN Bacterial Expression System, pTRE-Tight Vector, pTet-Off Vector, pTet-On Vector, Tet-Off Gene Expression System, Tet-On Gene Expression System, Adeno-X Tet-Off Expression System 1, Adeno-X Tet-On Expression System 1, Adeno-X Viral DNA (PI-Sce I/I-CeuI digested), Creator-Compatible RevTet-Off Retroviral Gene Expression System, Creator-Compatible RevTet-On Retroviral Gene Expression System, pLP-RevTRE Acceptor Vector, pRevTRE Vector, RevTet-Off System, RevTet-On System, pBI Tet Vector, pBI-G Tet Vector, pBI-L Tet Vector, pTet-tTS Vector, pLP-TRE2 Acceptor Vector, pTRE2 Vector, pTRE2hyg Vector, pTRE2pur Vector, Tet System Approved FBS, US-Sourced (Gamma Irradiated), pTRE-Tight-DsRed2 Vector and VP16 Minimal Domain Vector Set and Invitrogen vectors pTet-tTak and pTet-Splice. Reference is also made to U.S. Pat. Nos. 6,924,101, 6,905,836, 6,777,229, 6,759,236, 6,699,702, 6,699,692, 6,696,278, 6,680,301, 6,673,537, 6,667,150, 6,642,052, 6,620,618, 6,613,528, 6,544,782, 6,541,003, 6,503,748, 6,482,636, 6,440,741, 6,436,694, 6,410,317, 6,392,121, 6,376,192, 6,309,883, 6,303,383, 6,265,562, 6,261,807, 6,221,630, 6,218,181, 6,136,536, 6,127,171, 6,080,575, 6,033,856, 6,022,731, 6,010,875, 5,958,680, 5,955,363, 5,891,718, 5,869,035, 5,866,410, 5,858,762, 5,851,796, 5,849,576, 5,840,521, 5,830,727, 5,830,690, 5,786,162, 5,766,940, 5,637,503, 5,593,860, 5,585,254, 5,527,691, 5,516,669, 5,508,176, 5,384,259, 5,378,618, 5,348,886, 5,290,691, 5,256,568, 5,256,546, 5,166,070, 5,158,891, 5,151,364, 5,147,789, 5,053,335, 4,983,522, 4,879,230, 4,876,202, 4,874,703, 4,778,761, 4,752,574, 4,703,012, 4,680,260, 4,663,285, 4,634,677, 4,631,259, 4,581,335, 4,374,200 and 4,349,629.

The term plasmid covers any DNA transcription unit comprising a polynucleotide according to the invention and the elements necessary for its in vivo expression in a cell or cells of the desired host or target; and, in this regard, it is noted that a supercoiled or non-supercoiled, circular plasmid, as well as a linear form, are intended to be within the scope of the invention.

Each plasmid comprises or contains or consists essentially of, in addition to the polynucleotide encoding an antigen, epitope or immunogen, optionally fused with a heterologous peptide sequence, variant, analog or fragment, operably linked to a promoter or under the control of a promoter or dependent upon a promoter. In general, it is advantageous to employ a strong promoter functional in eukaryotic cells. The preferred strong promoter is the immediate early cytomegalovirus promoter (CMV-IE) of human or murine origin, or optionally having another origin such as the rat or guinea pig. The CMV-IE promoter can comprise the actual promoter part, which may or may not be associated with the enhancer part. Reference can be made to EP-A-260 148, EP-A-323 597, U.S. Pat. Nos. 5,168,062, 5,385,839, and 4,968,615, as well as to PCT Application No WO87/03905. The CMV-IE promoter is advantageously a human CMV-IE (Boshart M. et al., Cell., 1985, 41, 521-530) or murine CMV-IE.

In more general terms, the promoter has either a viral or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa M. et al., Vaccine, 2000, 18, 2337-2344), or the actin promoter (Miyazaki J. et al., Gene, 1989, 79, 269-277).

Functional sub fragments of these promoters, i.e., portions of these promoters that maintain an adequate promoting activity, are included within the present invention, e.g. truncated CMV-IE promoters according to PCT Application No. WO98/00166 or U.S. Pat. No. 6,156,567 can be used in the practice of the invention. A promoter in the practice of the invention consequently includes derivatives and sub fragments of a full-length promoter that maintain an adequate promoting activity and hence function as a promoter, preferably promoting activity substantially similar to that of the actual or full-length promoter from which the derivative or sub fragment is derived, e.g., akin to the activity of the truncated CMV-IE promoters of U.S. Pat. No. 6,156,567 to the activity of full-length CMV-IE promoters. Thus, a CMV-IE promoter in the practice of the invention can comprise or consist essentially of or consist of the promoter portion of the full-length promoter and/or the enhancer portion of the full-length promoter, as well as derivatives and sub fragments.

Preferably, the plasmids comprise or consist essentially of other expression control elements. It is particularly advantageous to incorporate stabilizing sequence(s), e.g., intron sequence(s), preferably the first intron of the hCMV-IE (PCT Application No. WO89/01036), the intron II of the rabbit β-globin gene (van Ooyen et al., Science, 1979, 206, 337-344).

As to the polyadenylation signal (polyA) for the plasmids and viral vectors other than poxviruses, use can more be made of the poly(A) signal of the bovine growth hormone (bGH) gene (see U.S. Pat. No. 5,122,458), or the poly(A) signal of the rabbit β-globin gene or the poly(A) signal of the SV40 virus.

The invention provides for the expression of any target peptide or polypeptide, advantageously an antigen, epitope, immunogen, peptide or polypeptide of interest by the DNA plasmids of the present invention. The invention contemplates the expression of any antigen, epitope, immunogen, peptide or polypeptide of interest in the DNA plasmids disclosed herein. The DNA plasmids can express one or more antigens, epitopes or immunogens of interest.

In an advantageous embodiment, the antigen, epitope, immunogen, peptide or polypeptide is derived from an avian, bovine, canine, equine, feline or porcine virus or pathogen. In another embodiment, the antigen, epitope, immunogen, peptide or polypeptide may be derived from West Nile virus. In another embodiment, the antigen, epitope, immunogen, peptide or polypeptide may be derived from a human virus or pathogen.

Avian antigens, epitopes or immunogens according to the invention can be derived from Marek's disease virus (MDV) (e.g., serotypes 1 and 2, preferably 1), Newcastle disease virus (NDV), Gumboro disease virus or infectious bursal disease virus (IBDV), infectious bronchitis virus (IBV), infectious anaemia virus or chicken anemia virus (CAV), infectious laryngotracheitis virus (ILTV), encephalomyelitis virus or avian encephalomyelitis virus (AEV or avian leukosis virus ALV), virus of hemorragic enteritis of turkeys (HEV), pneumovirosis virus (TRTV), fowl plague virus (avian influenza), chicken hydropericarditis virus, avian reoviruses, Escherichia coli, Mycoplasma gallinarum, Mycoplasma gallisepticum, Haemophilus avium, Pasteurella gallinarum, Pasteurella multocida gallicida, and mixtures thereof. Preferably, for MDV the immunogen is advantageously gB and/or gD, e.g., gB and gD, for NDV the immunogen is advantageously HN and/or F, e.g., HN and F; for IBDV the immunogen advantageously is VP2; for IBV the immunogen is advantageously S (more advantageously S1) and/or M and/or N, e.g., S (or S1) and M and/or N; for CAV the immunogen is advantageously VP1 and/or VP2; for ILTV the immunogen is advantageously gB and/or gD; for AEV the immunogen advantageously is env and/or gag/pro, e.g., env and gag/pro or gag/pro; for HEV the immunogen is advantageously the 100K protein and/or hexon; for TRTV the immunogen is advantageously F and/or G, and for fowl plague the immunogen is advantageously HA and/or N and/or NP, e.g., HA and N and/or NP.

Bovine antigens, epitopes or immunogens according to the invention can be derived from BHV-1, BRV, bPI-3 and/or BCV virus or a bovine pathogen selected from the group including but not limited to bovine respiratory syncytial virus and bovine viral diarrhea virus. BRSV immunogens can be BRSV F or G or N, such as BRSV F and/or G or N and/or G. BHV-1 immunogens can be gB and/or gC and/or gD. BVDV immunogens can be E0 the protein (gp48) and/or the E2 protein (gp53). The BVDV can be type 1 and/or type 2. The bPI-3 immunogens can be bPI-3 F and/or HN. See also U.S. Pat. Nos. 6,451,770, 6,376,473, 6,224,878, regarding immunogens of bovine pathogens and nucleic acid molecules coding therefor and constructs that express the same.

Canine antigens, epitopes or immunogens according to the invention can be derived from measles disease virus, canine distemper virus (CDV), canine parainfluenza type 2 virus (CPI-2), canine herpesvirus type 1 (CHV-1), rabies virus (rhabdovirus), canine parvovirus (CPV), canine coronavirus (CCV), canine adenovirus, Borrelia burgdorferi, Leptospira and mixtures thereof. Preferably, for CDV the immunogen is advantageously F and/or HA (see also U.S. Pat. Nos. 6,309,647, 5,756,102 regarding CDV immunogens and constructs); for CPV the immunogen is advantageously VP2; for CCV the immunogen is advantageously S and/or M; for CHV-1 the immunogen is advantageously gB and/or gC and/or gD (see also U.S. Pat. Nos. 5,688,920, 5,529,780, regarding CHV immunogens and constructs); for rabies virus the immunogen is advantageously G (see also U.S. Pat. No. 5,843,456 regarding rabies combination compositions); for Borrelia burgdorferi the immunogen is advantageously OspA (see also U.S. Pat. No. 6,368,603 regarding OspA combination compositions).

Equine antigens, epitopes or immunogens according to the invention can be derived from an EHV-1 and/or EHV-4 virus or another equine pathogen selected from the group including but not limited to equine influenza virus (EIV), eastern encephalomyelitis virus (EEV), western encephalomyelitis virus (WEV), Venezuelan encephalomyelitis virus (VEV), Lyme disease agent, Borrelia burgdorferi, Clostridium tetani, equine arteritis virus (EAV) and rabies virus. Antigens, epitopes or immunogens can be EHV glycoproteins such as gB, gD, gB+gD, gC, and gE, for EIV the immunogen is advantageously HA, NP and/or N; for viruses of encephalitis, the immunogen is advantageously C and/or E1 and/or E2; and for Clostridium tetani the immunogen is all or part of the subunit C of the tetanic toxin. Reference is made to U.S. Pat. Nos. 6,395,283, 6,248,333, 5,338,683, 6,183,750 for immunogens of equine pathogens and nucleic acid molecules coding therefor and consturcts that express the same.

Feline antigens, epitopes or immunogens according to the invention can be derived from feline herpesvirus type 1 (FHV-1), feline calicivirus (FCV), rabies virus (rhabdovirus), feline parvovirus (FPV), feline infectious peritonitis virus (FIPV), feline leukaemia virus (FeLV), feline immunodeficiency virus (FIV), Chlamydia and mixtures thereof. Preferably, for FeLV the immunogen is advantageously env and/or gag and/or pol, e.g., gag/pol; for FPV the immunogen is advantageously VP2; for FIPV the immunogen is advantageously S and/or M and/or N, e.g., S and M and/or N (see also U.S. Pat. Nos. 6,348,196 and 5,858,373 and immunogens and constructs thereof); for FHV the immunogen is advantageously gB and/or gC and/or gD, e.g., gB and gC and/or gD (see also U.S. Pat. Nos. 5,338,683, 6,183,750; for herpesvirus immunogens and constructs expressing the same); for FCV the immunogen is advantageously C; for FIV the immunogen is advantageously env and/or gag and/or pro, e.g., gag/pro, env, or env and gag/pro (see also immunogens and constructs discussed in Tartaglia et al., U.S. application Ser. No. 08/746,668, filed Nov. 14, 1996); for rabies virus the immunogen is advantageously G.

Porcine antigens, epitopes or immunogens according to the invention can be derived from PRRS virus and/or a porcine pathogen be selected from the group including but not limited to pseudorabies virus, porcine influenza virus, porcine parvovirus, transmissible gastro-enteritis virus (coronavirus), porcine circovirus such as porcine circovirus type 2, rotavirus, porcine adenovirus type 3, Escherichia coli, Erysipelothrix rhusiopathiae, Bordetella bronchiseptica, Clostridium spp., Salmonella spp., Haemophilus parasuis, Pasteurella multocida, Streptococcus suis, Mycoplasma hyopneumoniae and Actinobacillus pleuropneumoniae. Antigens, epitopes or immunogens of porcine pathogens can include pseudorabies virus gB, pseudorabies virus gC, pseudorabies virus gD, swine influenza HA, swine influenza NA, swine influenza NP, ORF4 of porcine reproductive and respiratory syndrome virus, ORF7 of porcine reproductive and respiratory syndrome virus, ORF5 of PRRSV, PRRSV ORF3, PRRSV ORF6, PRRSV open reading frames 5 (ORF5) and 6 (ORF6), PRRSV open reading frames 5 (ORF5) and 3 (ORF3) and 6 (ORF6), Hog Cholera Virus E1, Hog Cholera Virus E2 gene, parvovirus VP2, porcine circovirus type 2 ORF1, or porcine circovirus type 2 ORF2. Reference is made to U.S. Pat. Nos. 6,517,843, 6,497,883, 6,391,314, 6,379,676, 6,217,883, 6,207,165 and U.S. Patent publication 2003003112 and WO99/53047, WO99/08706, WO01/83737, and WO00/47756 for immunogens of porcine pathogens, nucleic acid molecules coding therefor and constructs expressing the same.

The DNA plasmids of the present invention can express one or more of the West Nile Virus (“WNV”) polynucleotides encoding E, prM, M or combinations or polyproteins thereof, (e.g., E, or E and prM, or E and M, or E and prM and M, or polyprotein E-prM-M, or polyprotein prM-E, or polyprotein M-E, or at least an epitope thereof. According to an embodiment of the invention, the other vector or vectors in the preparation comprises, consists essentially of or consists of a polynucleotide that encodes, and under appropriate circumstances the vector expresses one or more other proteins of the WN virus, e.g. prM, M, prM-M, or an epitope thereof.

As used herein, the term “antigen” or “immunogen” means a substance that induces a specific immune response in a host animal. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a protein, a polypeptide, a peptide, an epitope, a hapten, or any combination thereof. Alternately, the immunogen or antigen may comprise a toxin or antitoxin.

The term “immunogenic protein or peptide” as used herein also refers includes peptides and polypeptides that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. The term epitope relates to a protein site able to induce an immune reaction of the humoral type (B cells) and/or cellular type (T cells).

The term “immunogenic protein or peptide” further contemplates deletions, additions and substitutions to the sequence, so long as the polypeptide functions to produce an immunological response as defined herein. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide.

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

The terms “immunogenic” protein or polypeptide as used herein also refers to an amino acid sequence which elicits an immunological response as described above. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Methods especially applicable to the proteins of T. parva are fully described in the PCT Application Serial No. PCT/US2004/022605 incorporated herein by reference in its entirety.

Synthetic antigens are also included within the definition, for example, polyepitopes, flanking epitopes, and other recombinant antigens. See, e.g., Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998. Immunogenic fragments, for purposes of the present invention, will usually include at least about 3 amino acids, preferably at least about 5 amino acids, more preferably at least about 10-15 amino acids, and most preferably 25 or more amino acids, of the molecule. There is no critical upper limit to the length of the fragment, which could comprise nearly the full-length of the protein sequence, or even a fusion protein comprising at least one epitope of the protein.

Accordingly, a minimum structure of a polynucleotide expressing an epitope is that it comprises or consists essentially of or consists of nucleotides to encode an epitope or antigenic determinant of the protein or polyprotein of interest. A polynucleotide encoding a fragment of the total protein or polyprotein, more advantageously, comprises or consists essentially of or consists of a minimum of 21 nucleotides, advantageously at least 42 nucleotides, and preferably at least 57, 87 or 150 consecutive or contiguous nucleotides of the sequence encoding the total protein or polyprotein. Epitope determination procedures, such as, generating overlapping peptide libraries (Hemmer B. et al., Immunology Today, 1998, 19 (4), 163-168), Pepscan (Geysen et al., (1984) Proc. Nat. Acad. Sci. USA, 81, 3998-4002; Geysen et al., (1985) Proc. Nat. Acad. Sci. USA, 82, 178-182; Van der Zee R. et al., (1989) Eur. J. Immunol., 19, 43-47; Geysen H. M., (1990) Southeast Asian J. Trop. Med. Public Health, 21, 523-533; Multipin.®. Peptide Synthesis Kits de Chiron) and algorithms (De Groot A. et al., (1999) Nature Biotechnology, 17, 533-561), and in PCT Application Serial No. PCT/US2004/022605 all of which are incorporated herein by reference in their entireties, can be used in the practice of the invention, without undue experimentation. Other documents cited and incorporated herein may also be consulted for methods for determining epitopes of an immunogen or antigen and thus nucleic acid molecules that encode such epitopes.

A “polynucleotide” is a polymeric form of nucleotides of any length, which contain deoxyribonucleotides, ribonucleotides, and analogs in any combination. Polynucleotides may have three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double stranded form and each of two complementary forms known or predicted to make up the double stranded form of either the DNA, RNA or hybrid molecule.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The invention further comprises a complementary strand to a polynucleotide encoding an antigen, epitope, immunogen, peptide or polypeptide of interest. The complementary strand can be polymeric and of any length, and can contain deoxyribonucleotides, ribonucleotides, and analogs in any combination.

The terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

An “isolated” polynucleotide or polypeptide is one that is substantially free of the materials with which it is associated in its native environment. By substantially free, is meant at least 50%, advantageously at least 70%, more advantageously at least 80%, and even more advantageously at least 90% free of these materials.

Hybridization reactions can be performed under conditions of different “stringency.” Conditions that increase stringency of a hybridization reaction are well known. See for example, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The invention further encompasses polynucleotides encoding functionally equivalent variants and derivatives of the polypeptides of interest and functionally equivalent fragments thereof which may enhance, decrease or not significantly affect properties of the polypeptides encoded thereby. These functionally equivalent variants, derivatives, and fragments display the ability to retain biological activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the polynucleotide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp ://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

And, without undue experimentation, the skilled artisan can consult with many other programs or references for determining percent homology.

The invention further encompasses the polynucleotides of interest contained in a vector molecule or an expression vector and operably linked to a promoter element and optionally to an enhancer.

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of therapy, and may optionally be in the form of an expression cassette. As used herein, a vector needs not be capable of replication in the ultimate target cell or subject. The term includes cloning vectors also included are viral vectors.

The term “recombinant” means a polynucleotide semisynthetic, or synthetic origin which either does not occur in nature or is linked to another polynucleotide in an arrangement not found in nature.

“Heterologous” means derived from a genetically distinct entity from the rest of the entity to which it is being compared. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

The polynucleotides of the invention may comprise additional sequences, such as additional encoding sequences within the same transcription unit, controlling elements such as promoters, ribosome binding sites, polyadenylation sites, additional transcription units under control of the same or a different promoter, sequences that permit cloning, expression, homologous recombination, and transformation of a host cell, and any such construct as may be desirable to provide embodiments of this invention.

Elements for the expression of an antigen, epitope, immunogen, peptide or polypeptide of interest are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, e.g. a peptide, advantageously, in the vector, an ATG is placed at 5′ of the reading frame and a stop codon is placed at 3′. Other elements for controlling expression may be present, such as enhancer sequences, stabilizing sequences, such as intron and signal sequences permitting the secretion of the protein.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Serial No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312. The herein cited and incorporated herein by reference documents, in addition to providing examples of vectors useful in the practice of the invention, can also provide sources for peptides or fragments thereof to be expressed by vector or vectors in, or included in, the compositions of the invention.

The present invention also relates to preparations comprising vectors, such as expression vectors, e.g., therapeutic compositions. The preparations can comprise, consist essentially of, or consist of one or more vectors, e.g., expression vectors, such as in vivo expression vectors, comprising, consisting essentially or consisting of (and advantageously expressing) one or more of antigens, epitopes or immunogens of interest. Advantageously, the vector contains and expresses a polynucleotide that includes, consists essentially of, or consists of a polynucleotide coding for (and advantageously expressing) an antigen, epitope, immunogen, peptide or polypeptide of interest, in a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of an antigen, epitope, immunogen, peptide or polypeptide of interest or a fragment thereof.

According to another embodiment, the vector or vectors in the preparation comprise, or consist essentially of, or consist of polynucleotide(s) encoding one or more proteins or fragment(s) thereof of an antigen, epitope, immunogen, peptide or polypeptide of interest the vector or vectors expressing the polynucleotide(s). The inventive preparation advantageously comprises, consists essentially of, or consists of, at least two vectors comprising, consisting essentially of, or consisting of, and advantageously also expressing, advantageously in vivo under appropriate conditions or suitable conditions or in a suitable host cell, polynucleotides from different isolates encoding the same proteins and/or for different proteins, but advantageously the same proteins. Preparations containing one or more vectors containing, consisting essentially of or consisting of polynucleotides encoding, and advantageously expressing, advantageously in vivo, an antigen or fusion protein of interest or an epitope thereof.

The invention is also directed at mixtures of vectors that contain, consist essentially of, or consist of coding for, and express, different antigens, epitopes or immunogens, e.g., an antigen, epitope, immunogen, peptide or polypeptide from different species such as, but not limited to, birds, dogs, cats, cows, horses, humans and pigs.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof. Advantageous host cells include, but are not limited to, baby hamster kidney (BHK) cells, colon carcinoma (Caco-2) cells, COS7 cells, MCF-7 cells, MCF-10A cells, Madin-Darby canine kidney (MDCK) cells, Madin-Darby bovine kidnet cells (MDBK), mink lung (Mv1Lu) cells, MRC-5 cells, U937 cells and VERO cells, Chinese Hamster Ovary (CHO) Ki cells. Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, nucleofection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In an advantageous embodiment, the invention provides for the administration of a therapeutically effective amount of a formulation for the delivery and expression of an antigen, epitope, immunogen, peptide or polypeptide of interest in a target cell. Determination of the therapeutically effective amount is routine experimentation for one of ordinary skill in the art. In one embodiment, the formulation comprises an expression vector comprising a polynucleotide that expresses an antigen, epitope, immunogen, peptide or polypeptide of interest and a pharmaceutically or veterinarily acceptable carrier, vehicle or excipient. In an advantageous embodiment, the pharmaceutically or veterinarily acceptable carrier, vehicle or excipient facilitates transfection and/or improves preservation of the vector or protein.

The pharmaceutically or veterinarily acceptable carriers or vehicles or excipients are well known to the one skilled in the art. For example, a pharmaceutically or veterinarily acceptable carrier or vehicle or excipient can be a 0.9% NaCl (e.g., saline) solution or a phosphate buffer. Other pharmaceutically or veterinarily acceptable carrier or vehicle or excipients that can be used for methods of this invention include, but are not limited to, poly-(L-glutamate) or polyvinylpyrrolidone. The pharmaceutically or veterinarily acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description and can also be determined by the skilled artisan from this disclosure read in conjunction with the knowledge in the art, without any undue experimentation.

The cationic lipids containing a quaternary ammonium salt which are advantageously but not exclusively suitable for plasmids, are advantageously those having the following formula:

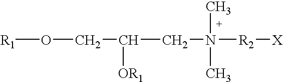

in which R₁ is a saturated or unsaturated straight-chain aliphatic radical having 12 to 18 carbon atoms, R₂ is another aliphatic radical containing 2 or 3 carbon atoms and X is an amine or hydroxyl group, e.g. the DMRIE. In another embodiment the cationic lipid can be associated with a neutral lipid, e.g. the DOPE.

Among these cationic lipids, preference is given to DMRIE (N-(2-hydroxyethyl)-N,N-dimethyl-2,3-bis(tetradecyloxy)-1-propane ammonium; WO96/34109), advantageously associated with a neutral lipid, advantageously DOPE (dioleoyl-phosphatidyl-ethanol amine; Behr J. P., 1994, Bioconjugate Chemistry, 5, 382-389), to form DMRIE-DOPE.

Advantageously, the plasmid mixture with the adjuvant is formed extemporaneously and advantageously contemporaneously with administration of the preparation or shortly before administration of the preparation; for instance, shortly before or prior to administration, the plasmid-adjuvant mixture is formed, advantageously so as to give enough time prior to administration for the mixture to form a complex, e.g. between about 10 and about 60 minutes prior to administration, such as approximately 30 minutes prior to administration.

When DOPE is present, the DMRIE:DOPE molar ratio is advantageously about 95:about 5 to about 5:about 95, more advantageously about 1:about 1, e.g., 1:1.

The DMRIE or DMRIE-DOPE adjuvant:plasmid weight ratio can be between about 50:about 1 and about 1:about 10, such as about 10:about 1 and about 1:about 5, and advantageously about 1:about 1 and about 1:about 2, e.g., 1:1 and 1:2.

The immunogenic compositions and vaccines according to the invention may comprise or consist essentially of one or more adjuvants. Suitable adjuvants for use in the practice of the present invention are (1) polymers of acrylic or methacrylic acid, maleic anhydride and alkenyl derivative polymers, (2) immunostimulating sequences (ISS), such as oligodeoxyribonucleotide sequences having one or more non-methylated CpG units (Klinman et al., Proc. Natl. Acad. Sci., USA, 1996, 93, 2879-2883; WO98/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, e.g., DDA (5) cytokines, (6) aluminum hydroxide or aluminum phosphate, (7) saponin or (8) other adjuvants discussed in any document cited and incorporated by reference into the instant application, or (9) any combinations or mixtures thereof.

The oil in water emulsion (3), which is especially appropriate for viral vectors, can be based on: light liquid paraffin oil (European pharmacopoeia type), isoprenoid oil such as squalane, squalene, oil resulting from the oligomerization of alkenes, e.g. isobutene or decene, esters of acids or alcohols having a straight-chain alkyl group, such as vegetable oils, ethyl oleate, propylene glycol, di(caprylate/caprate), glycerol tri(caprylate/caprate) and propylene glycol dioleate, or esters of branched, fatty alcohols or acids, especially isostearic acid esters. The oil is used in combination with emulsifiers to form an emulsion. The emulsifiers may be nonionic surfactants, such as: esters of on the one hand sorbitan, mannide (e.g. anhydromannitol oleate), glycerol, polyglycerol or propylene glycol and on the other hand oleic, isostearic, ricinoleic or hydroxystearic acids, said esters being optionally ethoxylated, or polyoxypropylene-polyoxyethylene copolymer blocks, such as Pluronic, e.g., L121.

Among the type (1) adjuvant polymers, preference is given to polymers of crosslinked acrylic or methacrylic acid, especially crosslinked by polyalkenyl ethers of sugars or polyalcohols. These compounds are known under the name carbomer (Pharmeuropa, vol. 8, no. 2, June 1996). One skilled in the art can also refer to U.S. Pat. No. 2,909,462, which provides such acrylic polymers crosslinked by a polyhydroxyl compound having at least three hydroxyl groups, preferably no more than eight such groups, the hydrogen atoms of at least three hydroxyl groups being replaced by unsaturated, aliphatic radicals having at least two carbon atoms. The preferred radicals are those containing 2 to 4 carbon atoms, e.g. vinyls, allyls and other ethylenically unsaturated groups. The unsaturated radicals can also contain other substituents, such as methyl. Products sold under the name Carbopol (BF Goodrich, Ohio, USA) are especially suitable. They are crosslinked by allyl saccharose or by allyl pentaerythritol. Among them, reference is made to Carbopol 974P, 934P and 971P.

As to the maleic anhydride-alkenyl derivative copolymers, preference is given to EMA (Monsanto), which are straight-chain or crosslinked ethylene-maleic anhydride copolymers and they are, for example, crosslinked by divinyl ether. Reference is also made to J. Fields et al., Nature 186: 778-780, Jun. 4, 1960.

With regard to structure, the acrylic or methacrylic acid polymers and EMA are preferably formed by basic units having the following formula:

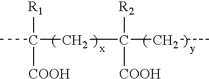

in which:
R₁ and R₂, which can be the same or different, represent H or CH₃
x=0 or 1, preferably x=1
y=1 or 2, with x+y=2.

For EMA, x=0 and y=2 and for carbomers x=y=1.

These polymers are soluble in water or physiological salt solution (20 g/l NaCl) and the pH can be adjusted to 7.3 to 7.4, e.g., by soda (NaOH), to provide the adjuvant solution in which the expression vector(s) can be incorporated. The polymer concentration in the final vaccine composition can range between 0.01 and 1.5% w/v, advantageously 0.05 to 1% w/v and preferably 0.1 to 0.4% w/v.

The cytokine or cytokines (5) can be in protein form in the immunogenic or vaccine composition, or can be co-expressed in the host with the immunogen or immunogens or epitope(s) thereof. Preference is given to the co-expression of the cytokine or cytokines, either by the same vector as that expressing the immunogen or immunogens or epitope(s) thereof, or by a separate vector therefor.

The invention comprehends preparing such combination compositions; for instance by admixing the active components, advantageously together and with an adjuvant, carrier, cytokine, and/or diluent.

Cytokines that may be used in the present invention include, but are not limited to, granulocyte colony stimulating factor (G-CSF), granulocyte/macrophage colony stimulating factor (GM-CSF), interferon α (IFN α), interferon β (IFN β), interferon γ, (IFN γ), interleukin-1α (IL-1α), interleukin-1β (IL-1β), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), tumor necrosis factor α (TNF α), tumor necrosis factor β (TNF β), and transforming growth factor β (TGF β). It is understood that cytokines can be co-administered and/or sequentially administered with the immunogenic or vaccine composition of the present invention. Thus, for instance, the vaccine of the instant invention can also contain an exogenous nucleic acid molecule that expresses in vivo a suitable cytokine, e.g., a cytokine matched to this host to be vaccinated or in which an immunological response is to be elicited (for instance, a canine cytokine for preparations to be administered to dogs).

Advantageously, the pharmaceutical and/or therapeutic compositions and/or formulations according to the invention comprise or consist essentially of or consist of an effective quantity to elicit a therapeutic response of one or more expression vectors and/or polypeptides as discussed herein; and, an effective quantity can be determined from this disclosure, including the documents incorporated herein, and the knowledge in the art, without undue experimentation.

In the case of therapeutic and/or pharmaceutical compositions based on a plasmid vector, a dose can comprise, consist essentially of or consist of, in general terms, about in 1 μg to about 2000 μg, advantageously about 50 μg to about 1000 μg and more advantageously from about 100 μg to about 800 μg of plasmid expressing the antigen, epitope, immunogen, peptide or polypeptide of interest. When the therapeutic and/or pharmaceutical compositions based on a plasmid vector is administered with electroporation the dose of plasmid is generally between about 0.1 μg and 1 mg, advantageously between about 1 μg and 100 μg, advantageously between about 2 μg and 50 μg. The dose volumes can be between about 0.1 and about 2 ml, advantageously between about 0.2 and about 1 ml. These doses and dose volumes are suitable for the treatment mammalian target species.

In an advantageous embodiment, an animal is vaccinated with two doses of inactivated vaccine at about 3 to 4 week intervals via the subcutaneous route, although an intramuscular route is also contemplated. Blood samples may be collected on the day of the first and/or second vaccination and about 2 to 4 weeks after the second vaccination to determine the levels of specific antibodies by methods known to one of skill in the art, for example, virus neutralization, hemagglutination inhibition, ELISA or single radial heamolysis (SRH) tests.

It should be understood by one of skill in the art that the disclosure herein is provided by way of example and the present invention is not limited thereto. From the disclosure herein and the knowledge in the art, the skilled artisan can determine the number of administrations, the administration route, and the doses to be used for each injection protocol, without any undue experimentation.

The present invention contemplates at least one administration to an animal of an efficient amount of the therapeutic composition made according to the invention. The animal may be male, female, pregnant female and newborn. This administration may be via various routes including, but not limited to, intramuscular (IM), intradermal (ID) or subcutaneous (SC) injection or via intranasal or oral administration. The therapeutic composition according to the invention can also be administered by a needleless apparatus (as, for example with a Pigjet, Biojector or Vitajet apparatus (Bioject, Oreg., USA)). Another approach to administer plasmid compositions is to use electroporation (see, e.g. S. Tollefsen et al. Vaccine, 2002, 20, 3370-3378; S. Tollefsen et al. Scand. J. Immunol., 2003, 57, 229-238; S. Babiuk et al., Vaccine, 2002, 20, 3399-3408; PCT Application No. WO99/01158). In another embodiment, the therapeutic composition is delivered to the animal by gene gun or gold particle bombardment. In an advantageous embodiment, the animal is a vertebrate.

One embodiment of the invention is a method of eliciting an immune response against the antigen, epitope, immunogen, peptide or polypeptide of interest in an animal, comprising administering a formulation for delivery and expression of a recombinant vaccine in an effective amount for eliciting an immune response. Still another embodiment of the invention is a method of inducing an immunological or protective response in an animal, comprising administering to the animal an effective amount of a formulation for delivery and expression of an antigen, epitope, immunogen, peptide or polypeptide of interest wherein the formulation comprises a recombinant vaccine and a pharmaceutically or veterinarily acceptable carrier, vehicle or excipient.

The invention relates to a method to elicit, induce or stimulate the immune response of an animal, advantageously a mammal or a vertebrate.

Another embodiment of the invention is a kit for performing a method of inducing an immunological or protective response against antigen, epitope, immunogen, peptide or polypeptide of interest in an animal comprising a recombinant vaccine and instructions for performing the method of delivery in an effective amount for eliciting an immune response in the animal.

The invention will now be further described by way of the following non-limiting examples.

### EXAMPLE 1

### Impact of Kanamycin Resistance (“Kanar”) Gene Expression Efficiency on Plasmid Replication Efficiency in Bacteria

The following example illustrates the impact of Kanamycin resistance (“KanaR”) gene expression efficiency on plasmid replication efficiency in bacteria.

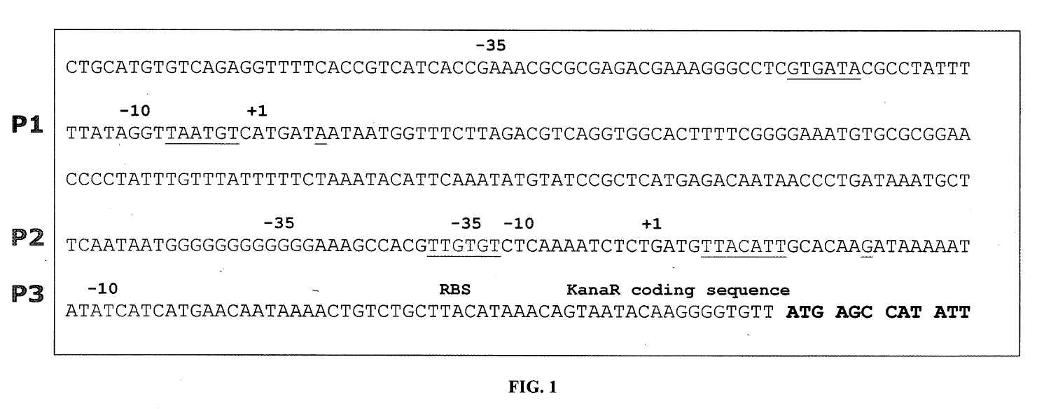

First, the KanaR expression cassette elements were analyzed, specifically promoter(s), translation initiation and transcription termination. Upon analysis of these expression elements, these elements are modified and the impact of the modified elements on plasmid production yields are analyzed. Four potential promoters were identified. P3 (SEQ ID NO: 3) was identified from database and literature searches, the structure being described in GenBank AN# X00928 (SEQ ID NO: 4) but not detected by current softwares. P2 (SEQ ID NO: 2) was determined by bioinformatic analysis and was also found in the published GenBank AN# V00359 (SEQ ID NO: 5). P1 (SEQ ID NO: 1) was an additional potential promoter belonging to the pVR1012 backbone. P0 was another potential promoter belonging to the pVR1012 backbone. FIG. 1 shows how P2 (SEQ ID NO: 2) and P3 partially overlap.

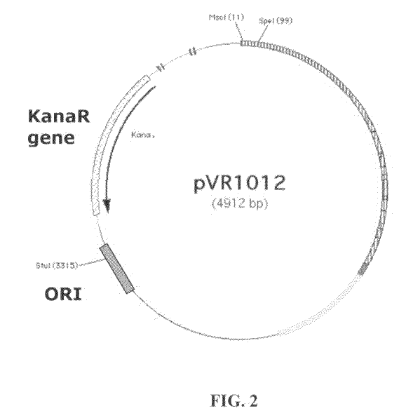

There is no transcription termination signal sequence for KanaR in pVR1012. This could have many potential consequences. Many KanaR transcripts could be abnormally long and degraded as there would be a useless metabolic burden. Moreover, KanaR expression could be sub-optimal in pVR1012. KanaR transcription may continue through the replication origin, ORI, and interfere with replication initiation as shown schematically in FIG. 2. Therefore, two appropriate transcription terminators were identified: rrnBT1+T2, which can be found in GenBank AN# U13872 (SEQ ID NO: 6) and is used in many commercial expression vectors, and speA, which has Acc. Number M31770 (SEQ ID NO: 7). Furthermore, the ATG initiator codon of pVR1012 was replaced by TTG in order to lower translation initiation efficacy. Thus, the aim was to determine the effect of each modified expression cassette on the kanamycin resistance capacity and plasmid production yields.

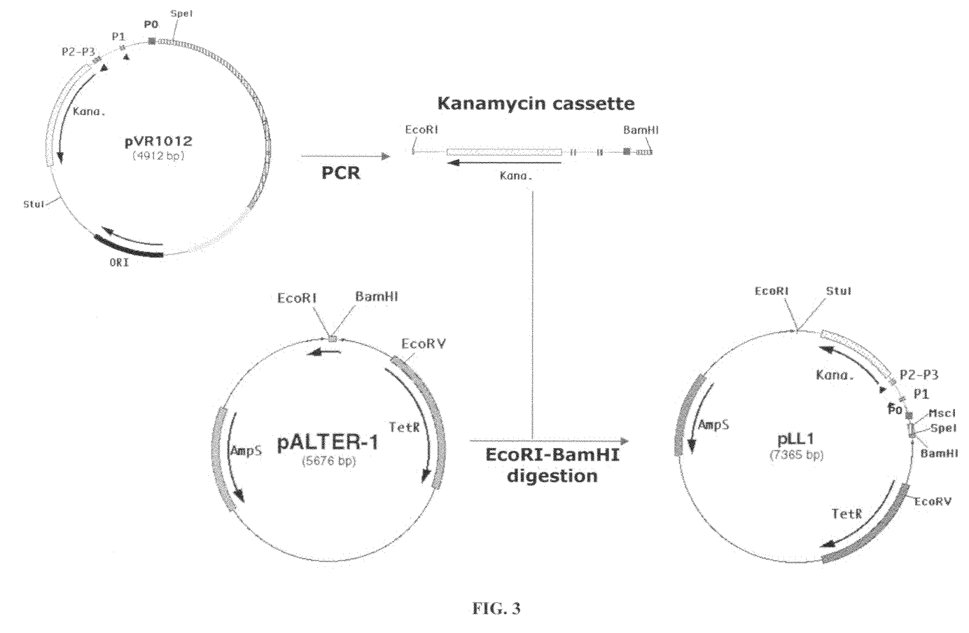

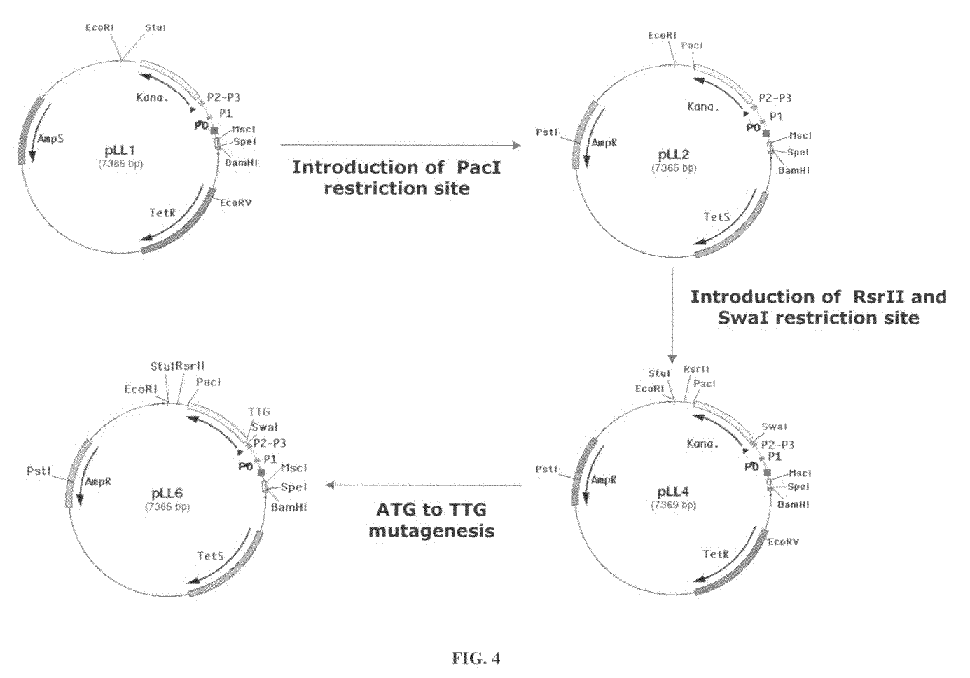

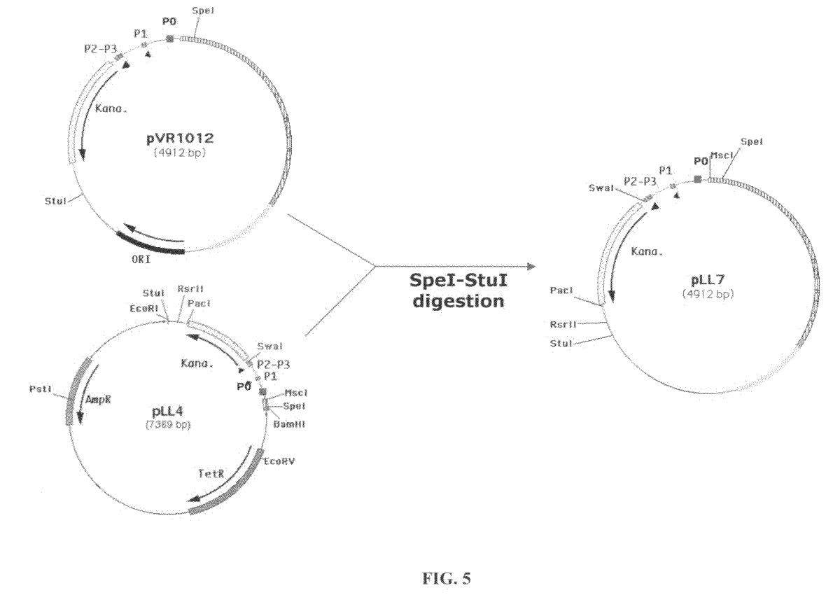

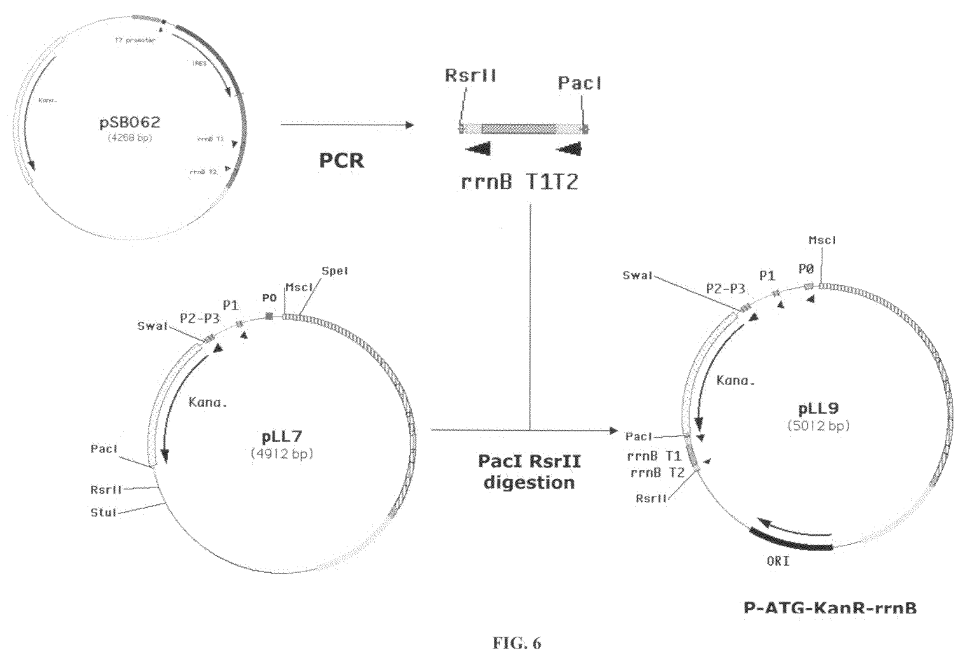

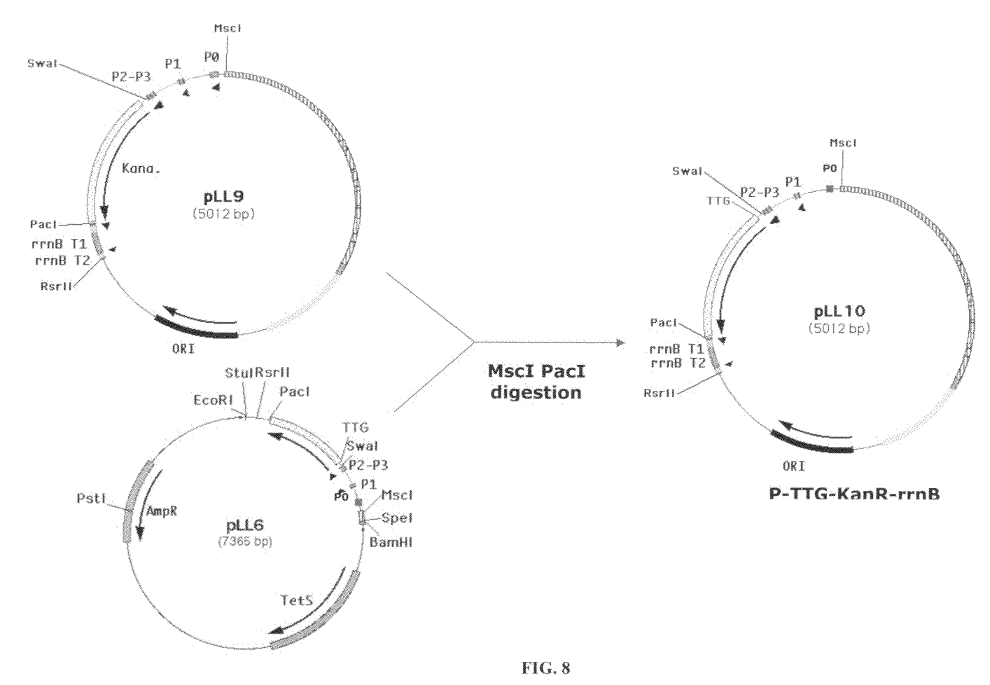

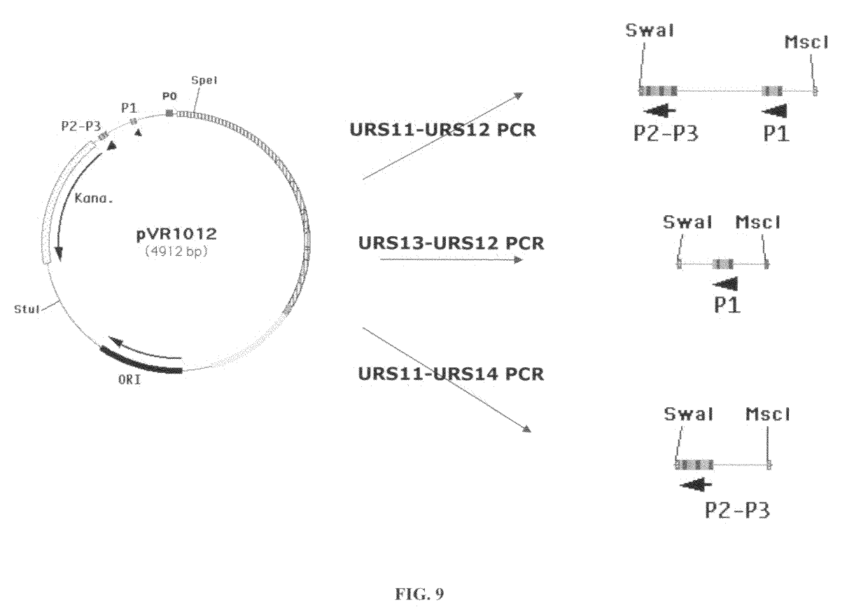

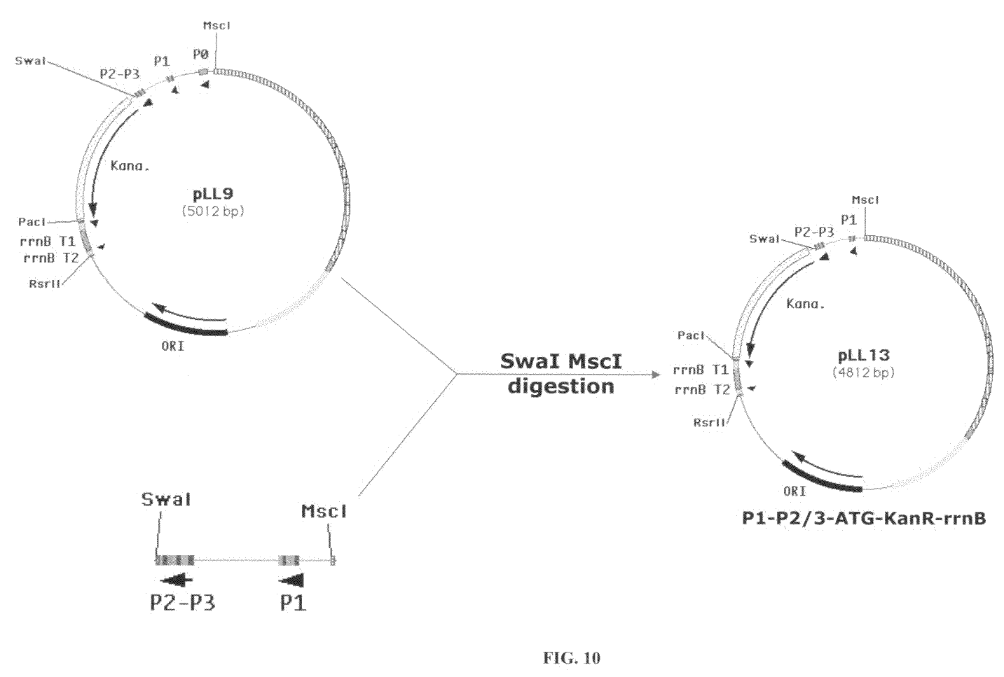

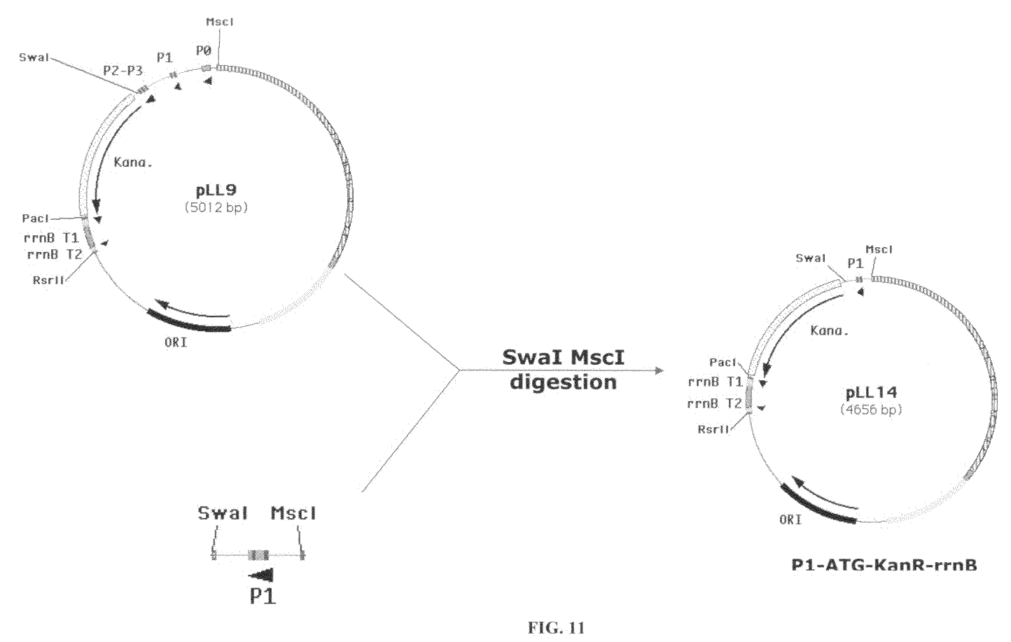

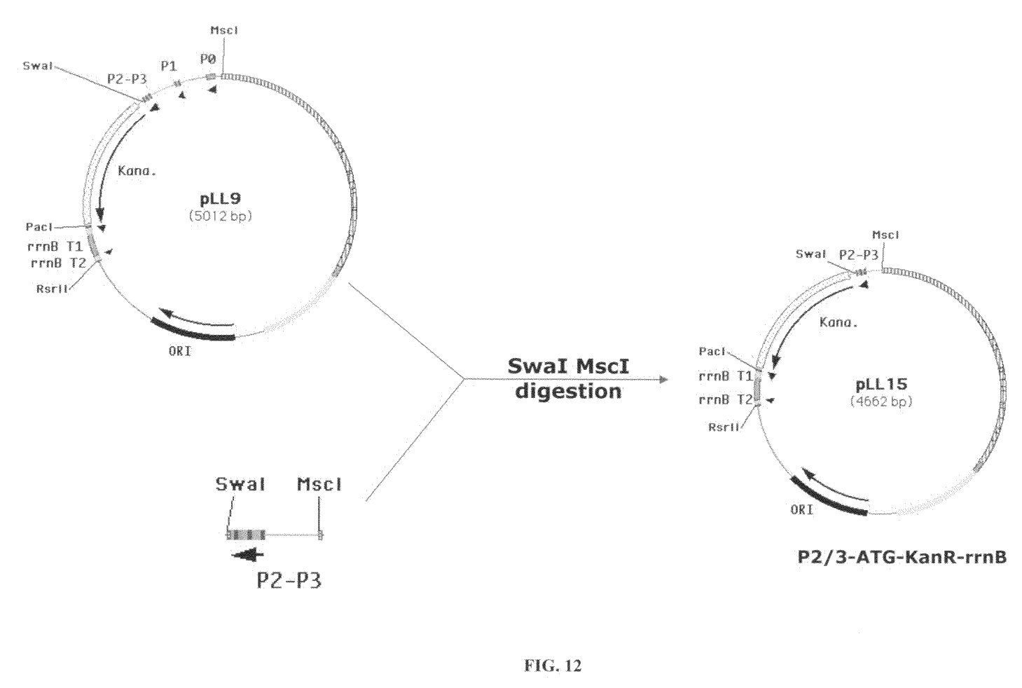

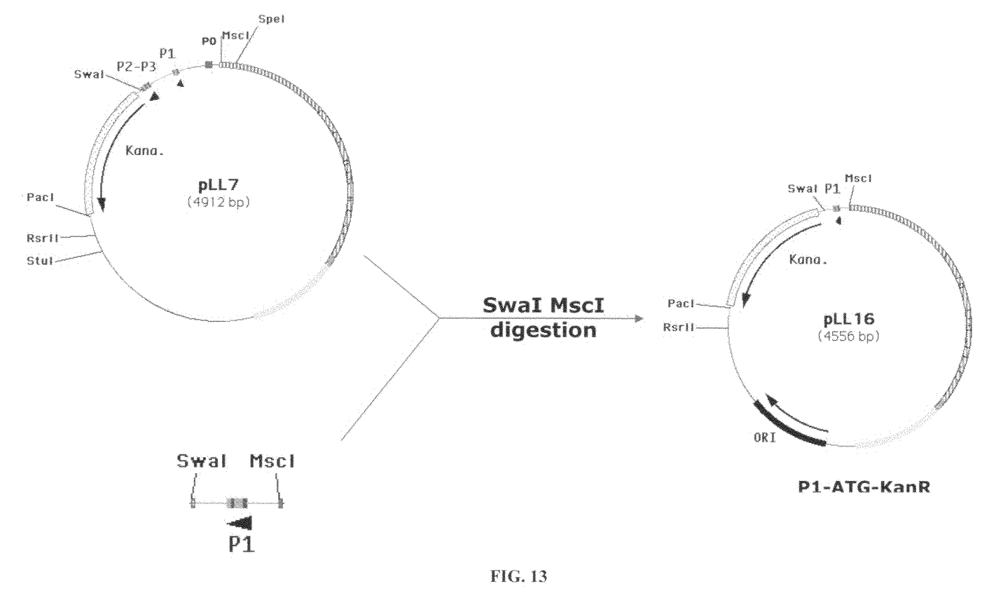

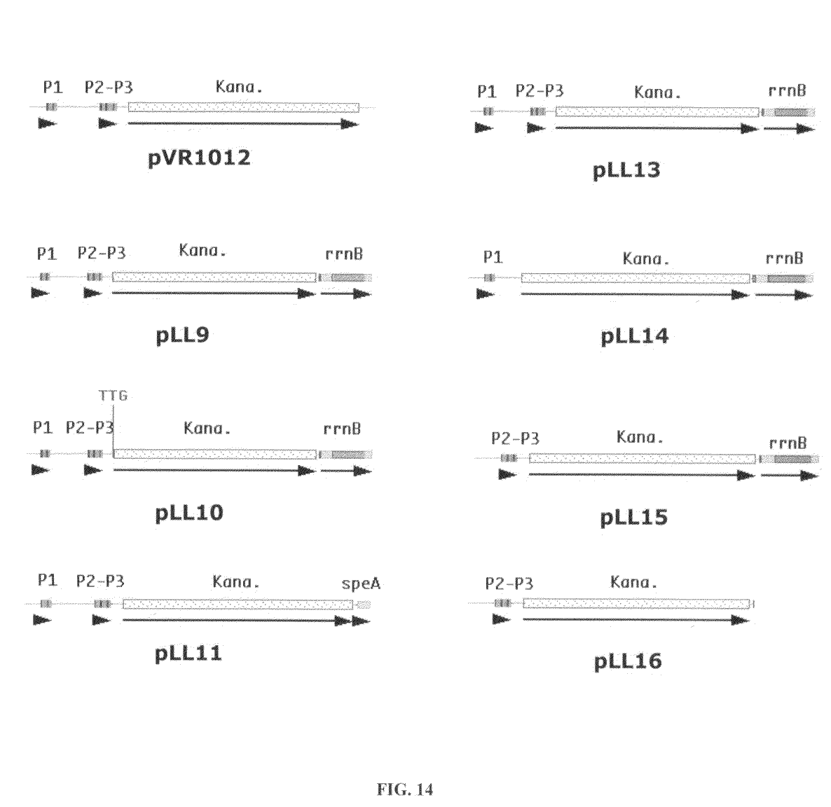

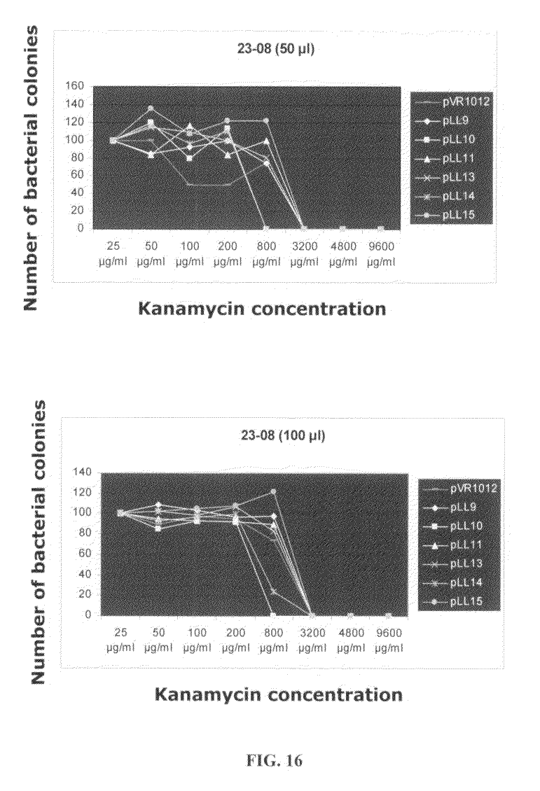

Thus, pVR1012 derivatives were generated with promoter P1 (SEQ ID NO: 1), P2/P3 (SEQ ID NOS: 2-3), or P1 (SEQ ID NO: 1) with P2/P3 (SEQ ID NOS: 2-3), transcription terminators rrnBT1+T2 or speA, and initiator codon TTG. In order to introduce such DNA sequences upstream or downstream the KanaR ORF, single restriction sites were needed. Such sites were not found in pVR1012 so they were introduced by site-directed mutagenesis. First, the KanaR cassette was cloned into the mutagenesis vector pALTER-1, as shown in FIG. 3. FIG. 4 shows how a PacI restriction site was introduced downstream of the KanaR ORF thereby producing pLL2, a SwaI restriction site was introduced upstream and a RsrII restriction site was introduced downstream thereby producing pLL4, and finally the translation initiator codon ATG was mutated to TTG, producing pLL6. The mutated KanaR cassette of pLL4 was then cloned into pVR1012 to form pLL7, as shown in FIG. 5. The rrnB terminator sequence was then cloned into pLL7 by PCR from pSB062 and subsequent PacI RsrII digestion to form pLL9, as shown in FIG. 6. FIG. 7 shows the cloning of the speA terminator sequence into pLL7 by annealing of synthetic oligonucleotides and PacI RsrII digestion to form pLL11. Next, the mutated translation initiation sequence of pLL6 was cloned into pLL9 by MscI PacI digestion to form pLL10, shown in FIG. 8. Finally, FIG. 9 illustrates how promoter sequences P2/P3 (SEQ ID NOS: 2-3) with P1 (SEQ ID NO: 1), P1 (SEQ ID NO: 1), and P2/P3 (SEQ ID NOS: 2-3) were cloned by URS11-URS12 PCR, URS13-URS12 PCR and URS11-URS14 PCR, respectively. FIGS. 10, 11 and 12 show how these were then incorporated into the pLL9 to form pLL13, 14 and 15, respectively. Similarly, FIG. 13 shows how P1 (SEQ ID NO: 1) was incorporated into pLL7 to form pLL16. FIG. 14 shows several of the resulting constructs. The pLL13, pLL14, pLL15 and pLL16 constructs had a 192 base pair deletion between the CMV promoter and the Kana P1 (SEQ ID NO: 1) promoter as illustrated by FIG. 15. Table 1 summarizes these constructs.

TABLE 1

| | KanaR gene | | | | |
|---|---|---|---|---|---|
| | | Initiator | | | Plasmid size |
| PLASMID | Promoter(s) | codon | Terminator | Insert | (bp) |
| pVR1012 | P0, P1, P2/3 | ATG | no | no | 4911 |
| pLL7 | P0, P1, P2/3 | ATG | no | no | 4911 |
| pLL9 | P0, P1, P2/3 | ATG | rrnB T1-T2 | no | 5011 |
| pLL10 | P0, P1, P2/3 | TTG | rrnB T1-T2 | no | 5011 |
| pLL11 | P0, P1, P2/3 | ATG | speA | no | 4846 |
| pLL13* | P1, P2/3 | ATG | rrnB T1-T2 | no | 4811 |
| pLL14* | P1 | ATG | rrnB T1-T2 | no | 4655 |
| pLL15* | P2/3 | ATG | rrnB T1-T2 | no | 4661 |
| pLL16* | P1 | ATG | no | no | 4555 |
| pPB662 | P0, P1, P2/3 | ATG | no | FIV env | 7456 |
| pLL19* | P1 | ATG | rrnB T1-T2 | FIV env | 7204 |

*Constructs having a 192 bp deletion between the CMV promoter and the Kana P1 promoter

The effect of the modifications on the KanaR gene transcript size was confirmed by Northern blot experiments. These confirmed the heterogeneity of pVR1012 KanaR transcripts as shown by the smear. They also confirmed the presence of three active promoters in pLL9, pLL10 and pLL11: one major short transcript from P2/P3 (SEQ ID NOS: 2-3) and two weaker transcripts from P0 and P1 (SEQ ID NO: 1). As expected, pLL13 generated two transcripts and pLL14 and pLL15 only one.

Several parameters were then tested on the pVR1012 derivatives in order to assess their new characteristics. The ability to resist increasing Kanamycin concentrations was tested by first inoculating one bacterial colony containing each construct, pVR1012 to pLL15, in LB medium and growing overnight at 37° C. and 200 RPM. Bacteria in each suspension were quantified by spectrophotometry (OD₆₀₀ nm). Appropriate dilutions of bacterial suspensions, 50 or 100 μl, were plated on LB medium containing increasing Kanamycin concentrations. Finally, colonies were counted, the results of which are shown in FIG. 16. These results showed that bacteria containing most pVR1012 derivatives had the same capacity to resist increasing Kana concentration without adaptation as growth was not affected between 0 and 800 μg/ml and was inhibited at 3200 μg/ml. Bacteria containing pLL10 and pLL14 had a significantly lowered capacity to resist Kanamycin as growth was 90% to 100% inhibited at 800 μg/ml, but not at 200 μg/ml. Thus, it was concluded that the absence of P2/P3 (SEQ ID NOS: 2-3) promoters or lowered translation efficiencies has a significant impact on the KanaR enzyme production, indicating that P2/P3 (SEQ ID NOS: 2-3) promoters are most efficient for KanaR expression and the TTG inhibitor lowers KanaR expression. However, these mutations still enable bacterial resistance and growth in the presence of classical Kana concentrations.

EGLI medium adaptation capacity was tested by first growing bacterial cell suspensions containing each bacterial construct and 10⁴ CFU in LB, and thereafter plating on EGLI plates. 25 to 50 isolated colonies containing each construct were subsequently plated on a new EGLI plate. The growing colonies were counted, the results of which are shown in Table 2. The pLL10, pLL14 and pLL16 plasmid backbones confer a clear growth advantage in nutrient-limited medium to bacteria containing them. Furthermore, lower Kanamycin production capacity is associated to better adaptation capacity on low nutrient medium in the presence of Kana 100 μg/ml.

TABLE 2

| | | Growth on EGLI plates | | Growth in EGLI broth | | Plasmid integrity | | |
|---|---|---|---|---|---|---|---|---|
| | | Nb. of colonies | | Nb. Of clones | | Number | Percentage | |
| | | grown/Nb of | percentage | grown/Nb of | percentage | of mutant | of mutant | Mutant |
| | Construct | colonies plated | growth | clones inoculated | growth | detected | detected | reference |
| Experiment 1 | pVR1012-I | 20/30 | 67 | 15/15 | 100 | 15/15 | 100 | all |
| | pLL9 | 22/30 | 73 | 15/15 | 100 | 3/13 | 23 | pLL9#3, #4 |
| | | | | | | | | and #15 |
| | pLL10 | 40/40 | 100 | 15/15 | 100 | 0/15 | 0 | — |
| | pLL11 | 36/40 | 90 | 11/15 | 73 | 1/11 | 9 | pLL11#3 |
| | pLL13 | 34/40 | 85 | 8/15 | 53 | 3/8 | 38 | pLL13#1, #3 |
| | | | | | | | | and #11 |
| | pLL14 | 30/30 | 100 | 15/15 | 100 | 0/15 | 0 | — |
| | pLL15 | 45/50 | 90 | 13/15 | 87 | 2/13 | 15 | pLL15#3 |
| | | | | | | | | and #14 |
| Experiment 2 | pVR1012-II | 11/25 | 44 | 6/11 | 55 | 1/6 | 17 | pVR1012#10 |
| | pLL16 | 25/25 | 100 | 25/25 | 100 | 0/25 | 0 | — |
| Experiment 3 | pVR1012-III | 25/25 | 100 | 25/25 | 100 | 9/25 | 36 | pVR1012#2, |
| | | | | | | | | #4, #8, #9, |
| | | | | | | | | #11, #13, |
| | | | | | | | | #14, #18 and |
| | | | | | | | | #24 |
| | pPB662 | 25/25 | 100 | 25/25 | 100 | 0/25 | 0 | — |
| | pLL14 | 25/25 | 100 | 25/25 | 100 | 0/25 | 0 | — |
| | pLL19 | 25/25 | 100 | 20/25 | 80 | 0/20 | 0 | — |

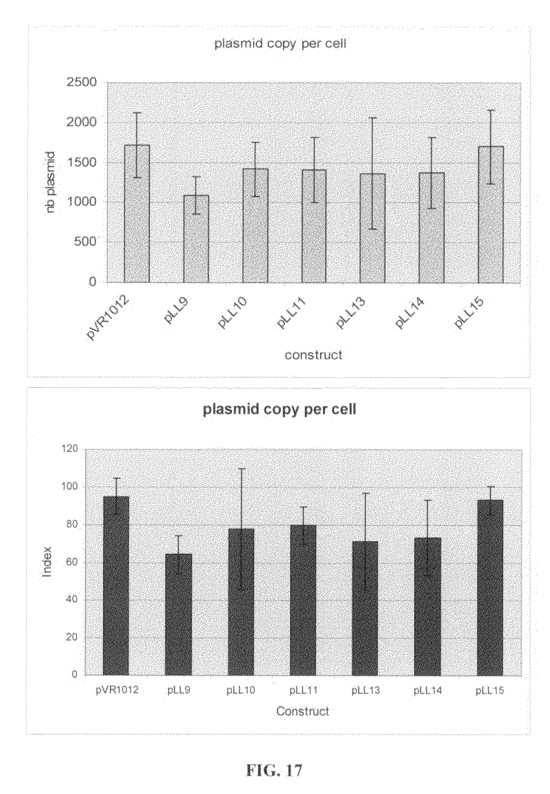

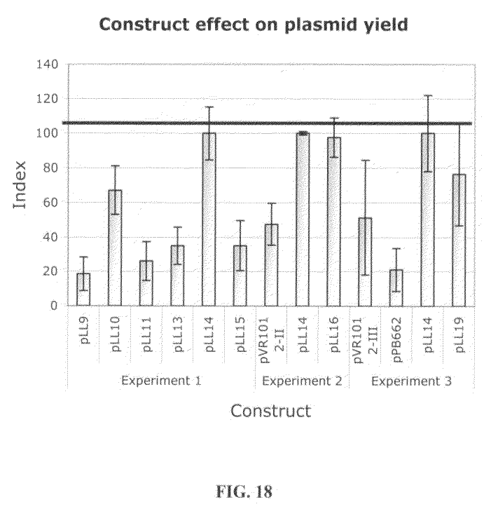

Plasmid yields obtained under laboratory growth conditions were tested by first growing one colony of bacteria containing each construct in liquid LB medium with Kana 100 μg/ml overnight at 37° C. and 200 RPM. The amount of bacteria in each suspension was evaluated by spectrophotometry, OD₆₀₀. DNA from each suspension was purified using Eppendorf columns and quantified by spectrophotometry, OD₂₆₀, each DNA solution being quantified twice. The number of plasmid copies in each suspension was then calculated, the results of which can be seen in FIG. 17. No significant differences were observed between the different constructs after growth in the LB medium with Kana 100 μg/ml. Plasmid stability was further evaluated by submitting three plasmid preparations derived from each construct to restriction analysis.

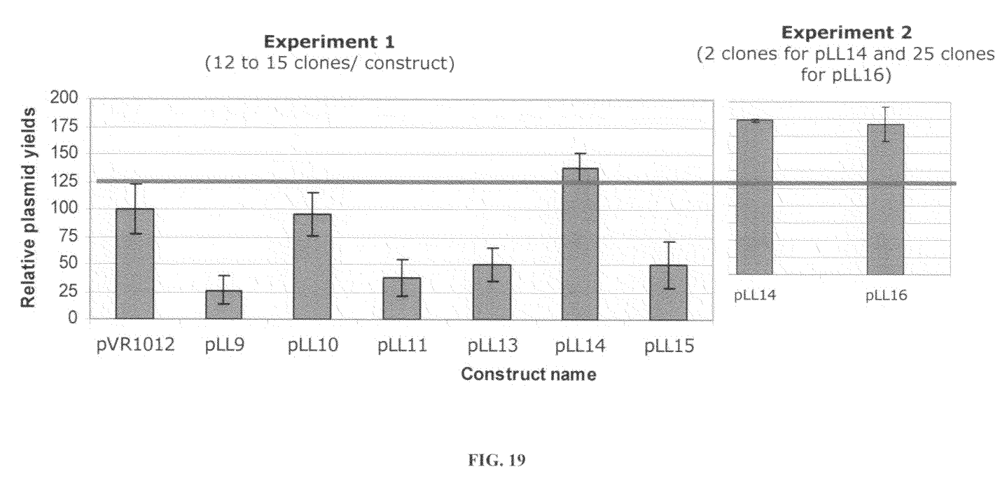

Plasmid yields obtained under laboratory growth conditions were tested by first adapting EGLI on solid medium. 15 and 25 colonies, Experiment A and B respectively, containing each construct were grown overnight in liquid EGLI medium at 37° C. and 200 RPM. The amount of bacteria in each suspension was evaluated by spectrophotometry, OD₆₀₀. DNA from each suspension was purified using Qiagen columns and quantified by spectrophotometry, OD₂₆₀. The number of plasmid copies in each suspension was then deduced, the results of which can be seen in FIG. 19.

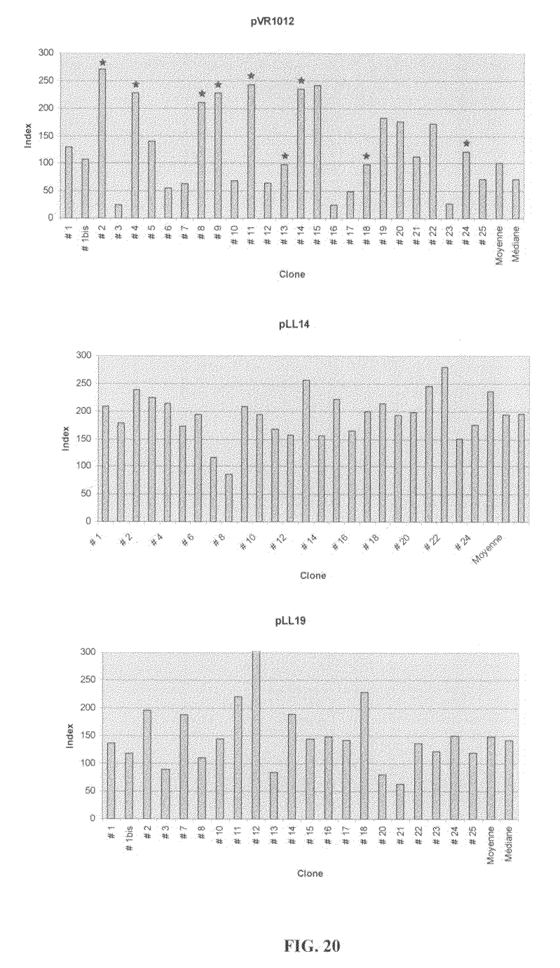

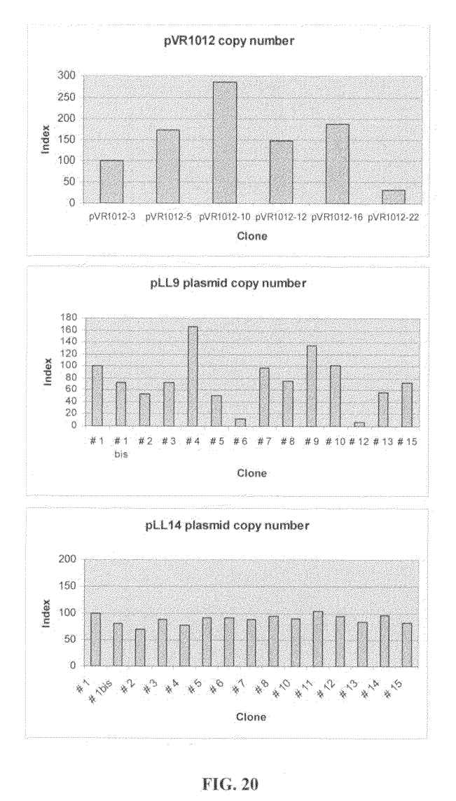

The results show that constructs pLL14 and pLL16 are the best candiates with respect to plasmid yields under laboratory conditions in EGLI medium. Furthermore, plasmid yields were significantly lower when constructs contained a transcription terminator and efficient translation initiation (ATG). Concomitantly, plasmid yields were highest when constructs did not contain a translation terminator or did contain the suboptimal P1 (SEQ ID NO: 1) promoter or suboptimal translation initiation (TTG). Plasmid yields were comparable in constructs pVR1012 and pLL10, indicating that low translation efficiency compensated the negative effect of the transcription terminator. These results suggest that the lower the capacity to express KanaR, the higher the plasmid yields are. In an extension of the test, plasmid stability was further evaluated by submitting each plasmid preparations derived from each construct to restriction analysis. Table 2 shows the results of this analysis. No mutation was detected after EGLI adaptation for pLL10, pLL14, pLL16 and pLL19 whereas it was more than 9% for the other constructs. It was also found that there was increased homogeneity of plasmid yields in pLL14, pLL16 and pLL19 EGLI-adapted clones. FIG. 20 illustrates the difference between the high heterogeneity of plasmid yield with constructs pVR1012 and a lower heterogeneity with construct pLL14 or pLL19.

Plasmid yields obtained by fermentation were tested by first arbitrarily selecting one clone of EGLI-adapted SCS1/pLL14 for fermentation. After fermentation, the bacteria were centrifuged and plasmid was extracted from an aliquot and quantified twice according to the methods developed by PGEA. The results showed that the growth rate of SCS1/pLL14 was significantly lower than that usually observed for pVR1012 derivatives, such as pPB266 and pPB662. Furthermore, the specific plasmid yield of pLL14 was of 3.1 mg plasmid/g of bacterial pellet. This result corresponds to the best results ever obtained with pVR1012 derivatives after Kana adaptation and clone selection, such as pPB266. Plasmid stability was then evaluated by submitting the plasmid preparations from each fermented clone to restriction analysis with three different restriction digestion patterns. Table 4 provides a summary of the overall plasmid analysis results. The results show that the level of KanaR expression had an impact on plasmid replication efficiency. The lower the expression, the higher replication rates were in nutrient-low medium and Kana 100 μg/ml. Lower KanaR expression could be obtained by deleting the natural promoter (P2/P3 (SEQ ID NOS: 2-3)), ensuring the absence of a transcription termination signal and mutating the translation initiation. Low KanaR expression led to up to 50% increased plasmid yields as compared to the parental plasmid backbone pVR1012. It also led to increased plasmid stability and homogeneity in plasmid production yields. This therefore probably allowed for limited clone selection.

TABLE 4

| | | | | Analysis | | | |
|---|---|---|---|---|---|---|---|
| | | | | Growth on | Plasmid yields | | |
| | | | | increasing | (“Erlen” | | Plasmid |
| Plasmid | | Transcrip. | Transl. | [Kana]: | growth in Egli/ | Mutation | yields |
| name | Promoter | terminator | init. | MIC | Kana 100) | rates | (fermentor) |
| pVR1012 | P1 + P2/3 | NO | ATG | 1600 to 3200 | 48** and 51*** | 17 to 100 | ND |
| pLL9 | P1 + P2/3 | rrnB T1-T2 | ATG | 1600 to 3200 | 19 | 23 | |
| pLL10 | P1 + P2/3 | rrnB T1-T2 | TTG | 400 to 800 | 67 | 0 | |
| pLL11 | P1 + P2/3 | SpeA | ATG | 1600 to 3200 | 26 | 9 | |
| pLL13* | P1 + P2/3 | rrnB T1-T2 | ATG | 1600 to 3200 | 35 | 38 | |
| pLL14* | P1 | rrnB T1-T2 | ATG | 400 to 800 | 100 | 0 | 3.1 mg/g of |
| | | | | | | | bacterial |
| | | | | | | | pellet |
| pLL15* | P2/3 | rrnB T1-T2 | ATG | 1600 to 3200 | 35 | 15 | ND |
| pLL16* | P1 | NO | ATG | | 98** | 0 | 1.9 mg/g of |
| | | | | | | | bacterial |
| | | | | | | | pellet |
| PLL19* | P1 | RrnB T1-T2 | ATG | — | 76*** | 0 | 2.9 mg/g of |
| | | | | | | | bacterial |
| | | | | | | | pellet |

*Constructs having a 192 bp deletion between the CMV promoter and the Kana P1 promoter.
**Results obtained in a second plasmid yield quantification test.
***Results obtained in a third plasmid yield quantification test.

Plasmid pLL16 is subject to further analysis, such as resistance to increasing Kana concentrations and plasmid yields after fermentation. Construction of pLL17 and pLL18 is also completed, wherein pLL17 has the P1 promoter, a TTG initiation codon and terminator rrnB T1/2 and pLL18 has the P1 promoter, a TTG initiation codon and no terminator. However, these constructs may be difficult to obtain due to low KanaR capacity. These constructions are analyzed using the previously described tests. Plasmid yields tests after Kana adaption on selected constructs (e.g., pLL14 to −18) are performed. RNA transcript analysis from the KanaR gene of all constructions is performed to confirm the deduced model. By flipping the KanaR cassette within the pVR1012, it can be confirmed that the KanaR cassette has no impact on the replication origin.

### EXAMPLE 2

### Sequences

| DEFINITION Transposon Tn2350 Km(r) gene 5′ region from plasmid R1. | |
|---|---|
| ACCESSION X00928 (SEQ ID NO: 4) | |
| 1 | gaattcccgc agattaacgc gcataacaag cggtttactc gttttggcct gcaatgtaac |
| 61 | ataatataca ttatgcgcac taaggtagag gcagcaagat tatgcggttt tgatcagaac |
| 121 | tcagttaacc atcccgggat tctgctctgg cccattcagc gcagtttttt actttggatg |
| 181 | aagttaaccc atgttatatt gcacaagata aaaatatatc atcatgaaca ataaaactgt |
| 241 | ctgcttacat aaacagtaat acaaggggtg ttatgagcca tattcaacgg gaaacgtctt |
| 301 | gctcgaggga attc |
| DEFINITION Transposon Tn903. | |
| ACCESSION V00359 J01839 (SEQ ID NO: 5) | |
| 1 | ggctttgttg aataaatcag atttcgggta agtctccccc gtagcgggtt gtgttttcag |
| 61 | gcaatacgca cgctttcagg catacctgct ttcgtcattt tgttcagcgc tcgtaccagg |
| 121 | gccatagcct ccgcaacctg accatcgtag tcacgcagcg tcagtgaacc cccgaacagc |
| 181 | tgttttaccc ggtacatcgc cgtttccgct atcgagcgac ggttgtaatc tgttgtccat |
| 241 | ttccaccgcg cattactccc ggtcattcgc tgattagcca ctgcacggtt acggtctgca |
| 301 | tattcaccgg gccagtaacc cgcacctttt cggggaggga taagcgcgct gattttctta |
| 361 | cgccgcagtt catcgtgaca gagccgggtg tcgtaagcgc cgtctgccga tgctgccctg |
| 421 | atttttctgt gagtctgccg gataagaccc gggaaggctt ctgagtcggt cacattgttc |
| 481 | agcgacaggt cagcgcagat gatttcatgt gttttactgt caacggcgag atgcagctta |
| 541 | cgccagatac ggcggcgttc ctggccatgc tttttgactt tccattcgcc ttcaccaaag |
| 601 | accttcagcc cggtggaatc aatcaccaga tgcgcgattt caccccgggt gaacgttttg |
| 661 | aaactgatat taaccgactt tgcccgcctg ctgacacagc tgtaatccgg gcagcgcaac |
| 721 | ggaacattca tcagtgtaaa aatggaatca ataaagccct gcgcagcgcg cagggtcagc |
| 781 | ctgaatacgc gtttaatgac cagcacagtc gtgatggcaa ggtcagaata gcgctgaggt |
| 841 | ctgcctcgtg aagaaggtgt tgctgactca taccaggcct gaatcgcccc atcatccagc |
| 901 | cagaaagtga gggagccacg gttgatgaga gctttgttgt aggtggacca gttggtgatt |
| 961 | ttgaactttt gctttgccac ggaacggtct gcgttgtcgg gaagatgcgt gatctgatcc |
| 1021 | ttcaactcag caaaagttcg atttattcaa caaagccacg ttgtgtctca aaatctctga |
| 1081 | tgttacattg cacaagataa aaatatatca tcatgaacaa taaaactgtc tgcttacata |
| 1141 | aacagtaata caaggggtgt tatgagccat attcaacggg aaacgtcttg ctcgaggccg |
| 1201 | cgattaaatt ccaacatgga tgctgattta tatgggtata aatgggctcg cgataatgtc |
| 1261 | gggcaatcag gtgcgacaat ctatcgattg tatgggaagc ccgatgcgcc agagttgttt |
| 1321 | ctgaaacatg gcaaaggtag cgttgccaat gatgttacag atgagatggt cagactaaac |
| 1381 | tggctgacgg aatttatgcc tcttccgacc atcaagcatt ttatccgtac tcctgatgat |
| 1441 | gcatggttac tcaccactgc gatccccggg aaaacagcat tccaggtatt agaagaatat |
| 1501 | cctgattcag gtgaaaatat tgttgatgcg ctggcagtgt tcctgcgccg gttgcattcg |
| 1561 | attcctgttt gtaattgtcc ttttaacagc gatcgcgtat ttcgtctcgc tcaggcgcaa |
| 1621 | tcacgaatga ataacggttt ggttgatgcg agtgattttg atgacgagcg taatggctgg |
| 1681 | cctgttgaac aagtctggaa agaaatgcat aagcttttgc cattctcacc ggattcagtc |
| 1741 | gtcactcatg gtgatttctc acttgataac cttatttttg acgaggggaa attaataggt |
| 1801 | tgtattgatg ttggacgagt cggaatcgca gaccgatacc aggatcttgc catcctatgg |
| 1861 | aactgcctcg gtgagttttc tccttcatta cagaaacggc tttttcaaaa atatggtatt |
| 1921 | gataatcctg atatgaataa attgcagttt catttgatgc tcgatgagtt tttctaatca |
| 1981 | gaattggtta attggttgta acactggcag agcattacgc tgacttgacg ggacggcggc |
| 2041 | tttgttgaat aaatcgaact tttgctgagt tgaaggatca gatcacgcat cttcccgaca |
| 2101 | acgcagaccg ttccgtggca aagcaaaagt tcaaaatcac caactggtcc acctacaaca |
| 2161 | aagctctcat caaccgtggc tccctcactt tctggctgga tgatggggcg attcaggcct |
| 2221 | ggtatgagtc agcaacacct tcttcacgag gcagacctca gcgctattct gaccttgcca |
| 2281 | tcacgactgt gctggtcatt aaacgcgtat tcaggctgac cctgcgcgct gcgcagggct |
| 2341 | ttattgattc catttttaca ctgatgaatg ttccgttgcg ctgcccggat tacagctgtg |
| 2401 | tcagcaggcg ggcaaagtcg gttaatatca gtttcaaaac gttcacccgg ggtgaaatcg |
| 2461 | cgcatctggt gattgattcc accgggctga aggtctttgg tgaaggcgaa tggaaagtca |
| 2521 | aaaagcatgg ccaggaacgc cgccgtatct ggcgtaagct gcatctcgcc gttgacagta |
| 2581 | aaacacatga aatcatctgc gctgacctgt cgctgaacaa tgtgaccgac tcagaagcct |
| 2641 | tcccgggtct tatccggcag actcacagaa aaatcagggc agcatcggca gacggcgctt |
| 2701 | acgacacccg gctctgtcac gatgaactgc ggcgtaagaa aatcagcgcg cttatccctc |
| 2761 | cccgaaaagg tgcgggttac tggcccggtg aatatgcaga ccgtaaccgt gcagtggcta |
| 2821 | atcagcgaat gaccgggagt aatgcgcggt ggaaatggac aacagattac aaccgtcgct |
| 2881 | cgatagcgga aacggcgatg taccgggtaa aacagctgtt cgggggttca ctgacgctgc |
| 2941 | gtgactacga tggtcaggtt gcggaggcta tggccctggt acgagcgctg aacaaaatga |
| 3001 | cgaaagcagg tatgcctgaa agcgtgcgta ttgcctgaaa acacaacccg ctacggggga |
| 3061 | gacttacccg aaatctgatt tattcaacaa agcc |
| DEFINITION pTrc99a cloning vector, complete sequence. | |
| ACCESSION U13872 (SEQ ID NO: 6) | |
| 1 | gtttgacagc ttatcatcga ctgcacggtg caccaatgct tctggcgtca ggcagccatc |
| 61 | ggaagctgtg gtatggctgt gcaggtcgta aatcactgca taattcgtgt cgctcaaggc |
| 121 | gcactcccgt tctggataat gttttttgcg ccgacatcat aacggttctg gcaaatattc |
| 181 | tgaaatgagc tgttgacaat taatcatccg gctcgtataa tgtgtggaat tgtgagcgga |
| 241 | taacaatttc acacaggaaa cagaccatgg aattcgagct cggtacccgg ggatcctcta |
| 301 | gagtcgacct gcaggcatgc aagcttggct gttttggcgg atgagagaag attttcagcc |
| 361 | tgatacagat taaatcagaa cgcagaagcg gtctgataaa acagaatttg cctggcggca |
| 421 | gtagcgcggt ggtcccacct gaccccatgc cgaactcaga agtgaaacgc cgtagcgccg |
| 481 | atggtagtgt ggggtctccc catgcgagag tagggaactg ccaggcatca aataaaacga |
| 541 | aaggctcagt cgaaagactg ggcctttcgt tttatctgtt gtttgtcggt gaacgctctc |
| 601 | ctgagtagga caaatccgcc gggagcggat ttgaacgttg cgaagcaacg gcccggaggg |
| 661 | tggcgggcag gacgcccgcc ataaactgcc aggcatcaaa ttaagcagaa ggccatcctg |
| 721 | acggatggcc tttttgcgtt tctacaaact ctttttgttt atttttctaa atacattcaa |
| 781 | atatgtatcc gctcatgaga caataaccct gataaatgct tcaataatat tgaaaaagga |
| 841 | agagtatgag tattcaacat ttccgtgtcg cccttattcc cttttttgcg gcattttgcc |
| 901 | ttcctgtttt tgctcaccca gaaacgctgg tgaaagtaaa agatgctgaa gatcagttgg |
| 961 | gtgcacgagt gggttacatc gaactggatc tcaacagcgg taagatcctt gagagttttc |
| 1021 | gccccgaaga acgttttcca atgatgagca cttttaaagt tctgctatgt ggcgcggtat |
| 1081 | tatcccgtgt tgacgccggg caagagcaac tcggtcgccg catacactat tctcagaatg |
| 1141 | acttggttga gtactcacca gtcacagaaa agcatcttac ggatggcatg acagtaagag |
| 1201 | aattatgcag tgctgccata accatgagtg ataacactgc ggccaactta cttctgacaa |
| 1261 | cgatcggagg accgaaggag ctaaccgctt ttttgcacaa catgggggat catgtaactc |
| 1321 | gccttgatcg ttgggaaccg gagctgaatg aagccatacc aaacgacgag cgtgacacca |
| 1381 | cgatgcctac agcaatggca acaacgttgc gcaaactatt aactggcgaa ctacttactc |
| 1441 | tagcttcccg gcaacaatta atagactgga tggaggcgga taaagttgca ggaccacttc |
| 1501 | tgcgctcggc ccttccggct ggctggttta ttgctgataa atctggagcc ggtgagcgtg |
| 1561 | ggtctcgcgg tatcattgca gcactggggc cagatggtaa gccctcccgt atcgtagtta |
| 1621 | tctacacgac ggggagtcag gcaactatgg atgaacgaaa tagacagatc gctgagatag |
| 1681 | gtgcctcact gattaagcat tggtaactgt cagaccaagt ttactcatat atactttaga |
| 1741 | ttgatttaaa acttcatttt taatttaaaa ggatctaggt gaagatcctt tttgataatc |
| 1801 | tcatgaccaa aatcccttaa cgtgagtttt cgttccactg agcgtcagac cccgtagaaa |
| 1861 | agatcaaagg atcttcttga gatccttttt ttctgcgcgt aatctgctgc ttgcaaacaa |
| 1921 | aaaaaccacc gctaccagcg gtggtttgtt tgccggatca agagctacca actctttttc |
| 1981 | cgaaggtaac tggcttcagc agagcgcaga taccaaatac tgtccttcta gtgtagccgt |
| 2041 | agttaggcca ccacttcaag aactctgtag caccgcctac atacctcgct ctgctaatcc |
| 2101 | tgttaccagt ggctgctgcc agtggcgata agtcgtgtct taccgggttg gactcaagac |
| 2161 | gatagttacc ggataaggcg cagcggtcgg gctgaacggg gggttcgtgc acacagccca |
| 2221 | gcttggagcg aacgacctac accgaactga gatacctaca gcgtgagcta tgagaaagcg |
| 2281 | ccacgcttcc cgaagggaga aaggcggaca ggtatccggt aagcggcagg gtcggaacag |
| 2341 | gagagcgcac gagggagctt ccagggggaa acgcctggta tctttatagt cctgtcgggt |
| 2401 | ttcgccacct ctgacttgag cgtcgatttt tgtgatgctc gtcagggggg cggagcctat |
| 2461 | ggaaaaacgc cagcaacgcg gcctttttac ggttcctggc cttttgctgg ccttttgctc |
| 2521 | acatgttctt tcctgcgtta tcccctgatt ctgtggataa ccgtattacc gcctttgagt |
| 2581 | gagctgatac cgctcgccgc agccgaacga ccgagcgcag cgagtcagtg agcgaggaag |
| 2641 | cggaagagcg cctgatgcgg tattttctcc ttacgcatct gtgcggtatt tcacaccgca |
| 2701 | tatggtgcac tctcagtaca atctgctctg atgccgcata gttaagccag tatacactcc |
| 2761 | gctatcgcta cgtgactggg tcatggctgc gccccgacac ccgccaacac ccgctgacgc |
| 2821 | gccctgacgg gcttgtctgc tcccggcatc cgcttacaga caagctgtga ccgtctccgg |
| 2881 | gagctgcatg tgtcagaggt tttcaccgtc atcaccgaaa cgcgcgaggc agcagatcaa |
| 2941 | ttcgcgcgcg aaggcgaagc ggcatgcatt tacgttgaca ccatcgaatg gtgcaaaacc |
| 3001 | tttcgcggta tggcatgata gcgcccggaa gagagtcaat tcagggtggt gaatgtgaaa |
| 3061 | ccagtaacgt tatacgatgt cgcagagtat gccggtgtct cttatcagac cgtttcccgc |
| 3121 | gtggtgaacc aggccagcca cgtttctgcg aaaacgcggg aaaaagtgga agcggcgatg |
| 3181 | gcggagctga attacattcc caaccgcgtg gcacaacaac tggcgggcaa acagtcgttg |
| 3241 | ctgattggcg ttgccacctc cagtctggcc ctgcacgcgc cgtcgcaaat tgtcgcggcg |
| 3301 | attaaatctc gcgccgatca actgggtgcc agcgtggtgg tgtcgatggt agaacgaagc |
| 3361 | ggcgtcgaag cctgtaaagc ggcggtgcac aatcttctcg cgcaacgcgt cagtgggctg |
| 3421 | atcattaact atccgctgga tgaccaggat gccattgctg tggaagctgc ctgcactaat |
| 3481 | gttccggcgt tatttcttga tgtctctgac cagacaccca tcaacagtat tattttctcc |
| 3541 | catgaagacg gtacgcgact gggcgtggag catctggtcg cattgggtca ccagcaaatc |
| 3601 | gcgctgttag cgggcccatt aagttctgtc tcggcgcgtc tgcgtctggc tggctggcat |
| 3661 | aaatatctca ctcgcaatca aattcagccg atagcggaac gggaaggcga ctggagtgcc |
| 3721 | atgtccggtt ttcaacaaac catgcaaatg ctgaatgagg gcatcgttcc cactgcgatg |
| 3781 | ctggttgcca acgatcagat ggcgctgggc gcaatgcgcg ccattaccga gtccgggctg |
| 3841 | cgcgttggtg cggatatctc ggtagtggga tacgacgata ccgaagacag ctcatgttat |
| 3901 | atcccgccgt taaccaccat caaacaggat tttcgcctgc tggggcaaac cagcgtggac |
| 3961 | cgcttgctgc aactctctca gggccaggcg gtgaagggca atcagctgtt gcccgtctca |
| 4021 | ctggtgaaaa gaaaaaccac cctggcgccc aatacgcaaa ccgcctctcc ccgcgcgttg |
| 4081 | gccgattcat taatgcagct ggcacgacag gtttcccgac tggaaagcgg gcagtgagcg |
| 4141 | caacgcaatt aatgtgagtt agcgcgaatt gatctg |
| DEFINITION E.coli arginine decarboxylase (speA) gene, complete cds, agmatinase | |
| (speB) and methionine adenosyltransferase (metK) genes, 5′ end. | |
| ACCESSION M31770 (SEQ ID NO: 7) | |
| 1 | tacccaaggt cgctggtggt gatttcgccg ccaactaaaa ccaatgccgg tttttacgta |
| 61 | ggtttcgcaa gcaacgcgtg ctttcggatc ctgttcgagg atcgcgtcta aaacggcatc |
| 121 | agaaatttgg tcagcaattt tgtcaggatg cccttcagag acggactcgg acgtaaaaag |
| 181 | gtgttttgcc atatttaata tcacctaaag agaatttggt tagctcaaac tgttgtgtgg |
| 241 | attttctgtg gtagcggatc ctaccacgac tctgcaggtt aaaaacactg gcagtctgag |
| 301 | tgttaatcgg tatggatgga ttaacatctg gatggctatt ttaggtcaat tcttcaccct |
| 361 | atttccactt ttttttgaat cgtgtctcat tctgttaaaa acgtggctgg aaatttttcc |
| 421 | tgacaatgcc ggcattctgc gtatttatct tttgcaattt tctgccattg tggggtataa |
| 481 | aacgcggcgc gcggcttaaa taaaaagcac acgacgtttc tttcgtgttg ccacttccag |
| 541 | ccgggttcaa atcagagttt tggcttgtgg gttcgtctta acaggcggcc gtggaggtga |
| 601 | tacgaaataa tgaaccgttg tctgctgctt aacctgtctc accgttctgg tgaagattcg |
| 661 | ttccccgcac tctgcatctc tgctttgcat acctgccgat gttataccca tctcggcgct |
| 721 | tctcaggatt caagagctgg ttacagttac tgaggactga acaagggcgc tcttgtaaaa |
| 781 | acaagagttt tctcgtggtt tcgccgaact ttcacactta cgttcggtta tgtgcttaat |
| 841 | aatgttatga aaaagaaacc ggttgcgcag ttggagcgtc agcattcact gctggaaaat |
| 901 | ccatgtgctt atgggttgtt atcgcagttc caggctgcga tagtcgttaa ctgttttaca |
| 961 | cttaataaaa taatttgagg ttcgctatgt ctgacgacat gtctatgggt ttgccttcgt |
| 1021 | cagcgggcga acacggtgta ctacgctcca tgcaggaggt tgcaatgagc tcccaggaag |
| 1081 | ccagcaagat gctgcgtact tacaatattg cctggtgggg caataactac tatgacgtta |
| 1141 | acgagctggg ccacattagc gtgtgcccgg acccggacgt cccggaagct cgcgtcgatc |
| 1201 | tcgcgcagtt agtgaaaact cgtgaagcac agggccagcg tctgcctgca ctgttctgtt |
| 1261 | tcccacagat cctgcagcac cgtttgcgtt ccattaacgc cgcgttcaaa cgtgcgaggg |
| 1321 | aatcctacgg ctataacggc gattacttcc ttgtttatcc gatcaaagtt aaccagcacc |
| 1381 | gccgcgtgat tgagtccctg attcattcgg gcgaaccgct gggtctggaa gccggttcca |
| 1441 | aagccgagtt gatggcagta ctggcacatg ctggcatgac ccgtagcgtc atcgtctgca |
| 1501 | acggttataa agaccgcgaa tatatccgcc tggcattaat tggcgagaag atggggcaca |
| 1561 | aggtctatct ggtcattgag aagatgtcag aaatcgccat tgtgctggat gaagcagaac |
| 1621 | gtctgaatgt cgttcctcgt ctgggcgtgc gtgcacgtct gcgttcgcag ggttcgggta |
| 1681 | aatggcagtc ctccggcggg gaaaaatcga agttcggcct ggctgcgact caggtactgc |
| 1741 | aactggttga aaccctgcgt gaagccgggc gtctcgacag cctgcaacta ctgcacttcc |
| 1801 | acctcggttc gcagatggcg aatattcgcg atatcgcgac aggcgttcgt gaatccgcgc |
| 1861 | gtttctatgt ggaactgcac aagctgggcg tcaatattca gtgcttcgac gtcggcggcg |
| 1921 | gtctgggcgt ggattatgaa ggtactcgtt cgcagtccga ctgttcggtg aactacggcc |
| 1981 | tcaatgaata cgccaacaac attatctggg cgattggcga tgcgtgtgaa gaaaacggtc |
| 2041 | tgccgcatcc gacggtaatc accgaatcgg gtcgtgcggt gactgcgcat cacaccgtgc |
| 2101 | tggtgtctaa tatcatcggc gtggaacgta acgaatacac ggtgccgacc gcgcctgcag |
| 2161 | aagatgcgcc gcgcgcgctg caaagcatgt gggaaacctg gcaggagatg cacgaaccgg |
| 2221 | gaactcgccg ttctctgcgt gaatggttac acgacagtca gatggatctg cacgacattc |
| 2281 | atatcggcta ctcttccggc atctttagcc tgcaagaacg tgcatgggct gagcagcttt |
| 2341 | atttgagcat gtgccatgaa gtgcaaaagc agctggatcc gcaaaaccgt gctcatcgtc |
| 2401 | cgattatcga cgagctgcag gaacgtatgg cggacaaaat gtacgtcaac ttctcgctgt |
| 2461 | tccagtcgat gccggacgca tgggggatcg accagttgtt cccggttctg ccgctggaag |
| 2521 | ggctggatca agtgccggaa cgtcgcgctg tgctgctgga tattacctgt gactctgacg |
| 2581 | gtgctatcga ccactatatt gatggtgacg gtattgccac gacaatgcca atgccggagt |
| 2641 | acgatccaga gaatccgccg atgctcggtt tctttatggt cggcgcatat caggagatcc |
| 2701 | tcggcaacat gcacaacctg ttcggtgata ccgaagcggt tgacgtgttc gtcttccctg |
| 2761 | acggtagcgt agaagtagaa ctgtctgacg aaggcgatac cgtggcggac atgctgcaat |
| 2821 | atgtacagct cgatccgaaa acgctgttaa cccagttccg cgatcaagtg aagaaaaccg |
| 2881 | atcttgatgc tgaactgcaa caacagttcc ttgaagagtt cgaggcaggt ttgtacggtt |
| 2941 | atacttatct tgaagatgag taagtcctgt gttacttgaa tccgcttaat ttagcggtga |
| 3001 | taatccgcca caatttattg tgacaaatcc aacccttcct cgtcgggcct aacgacgcgg |
| 3061 | aagggttttt ttatatcgac tttgtaatag gagtccatcc atgagcacct taggtcatca |
| 3121 | atacgataac tcactggttt ccaatgcctt tggtttttta cgcctgccga tgaacttcca |
| 3181 | gccgtatgac agcgatgcag actgggtgat tactggcgtg ccgttcgata tggcca |

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A DNA plasmid comprising a kanamycin resistance gene wherein
(a) the kanamycin resistance gene comprises the promoter of SEQ ID NO: 1 and/or a TTG initiation codon,
(b) the kanamycin resistance gene does not comprise the promoter of SEQ ID NO: 2, the promoter of SEQ ID NO: 3 and/or a transcriptional termination signal,
wherein the expression of the kanamycin resistance gene is decreased as compared to a kanamycin resistance gene comprising the promoter of SEQ ID NO: 2, the promoter of SEQ ID NO: 3 and/or a transcription terminal signal.

2. The DNA plasmid of claim 1 wherein the decreased kanamycin resistance gene expression results in higher plasmid yields and higher plasmid stability as compared to a DNA plasmid comprising a kanamycin resistance gene comprising the promoter of SEQ ID NO: 2, the promoter of SEQ ID NO: 3 and/or a transcription terminal signal.

3. The DNA plasmid of claim 1 or 2 wherein the kanamycin resistance gene comprises the promoter of SEQ ID NO: 1.

4. The DNA plasmid of any one of claims 1-3 wherein the kanamycin resistance gene comprises the TTG initiation codon.

5. The DNA plasmid of claim 1 wherein the DNA plasmid is pLL10, pLL14, pLL16 or pLL18.

6. A DNA plasmid comprising a kanamycin resistance gene wherein a transposon is inserted between the kanamycin resistance gene promoter and translation initiation, thereby resulting in decreased kanamycin resistance gene expression.

7. The DNA plasmid of claim 6 wherein the decreased kanamycin resistance gene expression results in higher plasmid yields and higher plasmid stability as compared to a DNA plasmid comprising a kanamycin resistance gene without a transposon inserted between the kanamycin resistance gene promoter and translation initiation.

8. A formulation for delivery and expression of an antigen, epitope, immunogen, peptide or polypeptide of interest, wherein the formulation comprises the plasmid of any one of claims 1-7 and a pharmaceutically or veterinarily acceptable carrier, vehicle or excipient.

9. The formulation of claim 8, wherein the carrier, vehicle or excipient facilitates transfection and/or improves preservation of the vector or protein.

10. The formulation of claim 8 or 9 wherein the antigen, epitope, immunogen, peptide or polypeptide of interest is derived from an avian, bovine, canine, equine, feline or porcine virus or pathogen.

11. A method of stimulating an immune response in an animal comprising administering an effective amount of the formulation of any one of claims 8-10 to cells of the animal and expressing the antigen, epitope, immunogen, peptide or polypeptide of interest in the cells.

12. A method of eliciting an immune response in an animal comprising administering an effective amount of the formulation of any one of claims 8-10 to cells of the animal and expressing the antigen, epitope, immunogen, peptide or polypeptide of interest in the cells.

13. The method of claim 11 or 13 wherein the animal is an avian, a bovine, a canine, an equine, a feline or a porcine.

14. A kit for performing any one of the methods of claims 11 to 13 comprising the DNA plasmid or formulation of any one of claims 1 to 10 and instructions for performing the method of any one of claims 11 to 13.
